(19) United States
(12) Patent Application Publication
(10) Pub. No.: US 2024/0400653 A1
(43) Pub. Date: Dec. 5, 2024

(54) ANTI-SARS-COV-2 ANTIBODIES AND USES THEREOF I

(71) Applicant: Seqirus Pty Ltd, Victoria (AU)

(72) Inventors: Steven ROCKMAN, Victoria (AU); Kirsten VANDENBERG, Victoria (AU); Chi ONG, Victoria (AU)

(21) Appl. No.: 18/699,102

(22) PCT Filed: Oct. 7, 2022
(86) PCT No.: PCT/AU2022/051202

§ 371 Date: Apr. 5, 2024

(30) Foreign Application Priority Data
Oct. 7, 2021 (AU) 2021903205

(51) Int. Cl. C07K 16/10

(52) U.S. Cl. CPC C07K 16/1003; C07K 2317/24; C07K 2317/33; C07K 2317/76; C07K 2317/92

(57) ABSTRACT

The present disclosure relates to proteins which bind to severe acute respiratory syndrome coronavirus 2 (SARS-COV-2) and uses thereof.

### RELATED APPLICATION DATA

The present application claims priority from Australian Patent Application No. 2021903205 filed 7 Oct. 2021 entitled “Anti-SARS-COV-2 Antibodies and Uses Thereof I”, the entire contents of which is hereby incorporated by reference.

### SEQUENCE LISTING

The present application is filed together with a Sequence Listing in electronic form. The entire contents of the Sequence Listing are hereby incorporated by reference.

### FIELD

The present disclosure relates to proteins which bind to severe acute respiratory syndrome coronavirus 2 (SARS-COV-2) and uses thereof.

### BACKGROUND

Respiratory viral infections are a significant threat to human health and lives. Infections, such as those caused by the severe acute respiratory syndrome coronavirus (SARS-COV) have been known to cause global pandemics, killing millions of people worldwide. In late 2019, a novel SARS virus (SARS-COV-2) was identified in China in humans and in March 2020 the World Health Organisation declared the outbreak of the virus a worldwide pandemic.

Human infection with SARS-COV-2 displays a broad clinical spectrum with a high rate of transmissibility. The global count of infections and mortality continues to rise with infections exceeding 615 million and over 6.54 million deaths to date.

The SARS-COV-2 pandemic has seen the unprecedented development of multiple vaccines, with 8 vaccines approved for full use and over 130 in Phase 1-3 clinical trials The majority of vaccines in development attempt to evoke the immune system to recognise the SARS-COV-2 spike protein (or S protein) since early studies of recombinant SARS-COV protein in a hamster challenge model demonstrated that this approach was immunogenic and protective. For those specific treatments available, for example, mRNA vaccines against COVID-19, further improvements can be made to increase their efficacy.

Therefore, it will be apparent to the skilled person that there is a need in the art for therapeutics against SARS-COV-2 and COVID-19.

### SUMMARY

The present disclosure is based on the inventors' identification of a protein against severe acute respiratory syndrome coronavirus 2 (SARS-COV-2). Specifically, the inventors have developed proteins comprising an antibody variable region that bind to a SARS-COV-2 spike(S) protein receptor binding domain (RBD) and neutralize binding of the S protein to angiotensin-converting enzyme 2 (ACE2), which is expressed on the surfaces of cells. The inventors have also identified that the neutralising proteins specifically bind a conformational, non-linear, epitope on the S protein.

Broadly, the findings of the inventors provide the basis for a SARS-COV-2 neutralising protein comprising an antibody variable region. The findings by the inventors also provide the basis for methods of treating, preventing and/or delaying progression of a disease or disorder (e.g., a disease caused by a SARS-COV-2 infection, such as COVID-19 or ARDS) in a subject. The findings by the inventors further provide the basis for detecting an antigen having a conformation sufficient to induce an immune response against SARS-COV-2.

Accordingly, the present disclosure provides a protein comprising an antibody variable region, wherein the antibody variable region binds to or specifically binds to a SARS-COV-2 S protein RBD and neutralises binding of the S protein to ACE2.

The present disclosure also provides a protein comprising an antibody variable region, wherein the antibody variable region binds to or specifically binds to a SARS-CoV-2 S protein RBD and neutralises binding of the S protein to ACE2 and wherein the antibody variable region competitively inhibits binding of any one of the following antibodies to the SARS-COV-2 S protein RBD:
(a) an antibody comprising a heavy chain variable region (V_{H}) comprising a sequence set forth in SEQ ID NO: 1 and a light chain variable region (V_{L}) comprising a sequence set forth in SEQ ID NO: 2;
(b) an antibody comprising a V_{H} comprising a sequence set forth in SEQ ID NO: 3 and a V_{L} comprising a sequence set forth in SEQ ID NO: 4;
(c) an antibody comprising a V_{H} comprising a sequence set forth in SEQ ID NO: 5 and a V_{L} comprising a sequence set forth in SEQ ID NO: 6;
(d) an antibody comprising a V_{H} comprising a sequence set forth in SEQ ID NO: 7 and a V_{L} comprising a sequence set forth in SEQ ID NO: 8; and
(e) an antibody comprising a V_{H} comprising a sequence set forth in SEQ ID NO: 9 and a V_{L} comprising a sequence set forth in SEQ ID NO: 10.

In one example, the antibody variable region competitively inhibits binding of an antibody comprising a V_{H} comprising a sequence set forth in SEQ ID NO: 1 and a V_{L} comprising a sequence set forth in SEQ ID NO: 2 to the SARS-COV-2 S protein RBD.

In one example, the antibody variable region competitively inhibits binding of an antibody comprising a V_{H} comprising a sequence set forth in SEQ ID NO: 3 and a V_{L} comprising a sequence set forth in SEQ ID NO: 4 to the SARS-COV-2 S protein RBD.

In one example, the antibody variable region competitively inhibits binding of an antibody comprising a V_{H} comprising a sequence set forth in SEQ ID NO: 5 and a V_{L} comprising a sequence set forth in SEQ ID NO: 6 to the SARS-COV-2 S protein RBD.

In one example, the antibody variable region competitively inhibits binding of an antibody comprising a V_{H} comprising a sequence set forth in SEQ ID NO: 7 and a V_{L} comprising a sequence set forth in SEQ ID NO: 8 to the SARS-COV-2 S protein RBD.

In one example, the antibody variable region competitively inhibits binding of an antibody comprising a V_{H} comprising a sequence set forth in SEQ ID NO: 9 and a V_{L} comprising a sequence set forth in SEQ ID NO: 10 to the SARS-COV-2 S protein RBD.

Methods of determining competitive inhibition will be apparent to the skilled person and/or are described herein.

Methods of determining neutralising activity of a protein will be apparent to the skilled person and/or are described herein. Exemplary assays include a Vero microneutralisation assay, a surrogate viral neutralisation test (sVNT) and a psuedovirus neutralisation assay (PsV; using e.g., 293T or HeLa-ACE2 cell lines). It will be apparent from the foregoing that the protein need not completely neutralize binding of a SARS-CoV-2 S protein to ACE2, rather it need only neutralize binding by a statistically significant amount, for example, by at least about 30%, or about 40%, or about 50%, or about 60%, or about 70%, or about 80%, or about 90%, or about 95%, or about 100%.

In one example, the protein neutralises binding of a SARS-COV-2 S protein to ACE2 in a sVNT assay. In one example, the protein neutralises binding of a SARS-CoV-2 S protein to ACE2 by about 30%, or about 40%, or about 50%, or about 60%, or about 70%, or about 80%, or about 90%, or about 95%, or about 100% in a sVNT assay. In one example, the protein neutralises binding of a SARS-COV-2 S protein to ACE2 by about 30% to about 50% in a sVNT assay. For example, by about 30% or about 35%, or about 40%, or about 45%, or about 50%. In one example, the protein neutralises binding of a SARS-COV-2 S protein to ACE2 by about 30% in a sVNT assay. In another example, the protein neutralises binding of a SARS-COV-2 S protein to ACE2 by about 40% in a sVNT assay. In a further example, the protein neutralises binding of a SARS-CoV-2 S protein to ACE2 by about 45% in a sVNT assay. In another example, the protein neutralises binding of a SARS-COV-2 S protein to ACE2 by about 50% in a sVNT assay. In a further example, the protein neutralises binding of a SARS-COV-2 S protein to ACE2 by about 50% to about 80% in a sVNT assay. For example, by about 50%, or about 55%, or about 60%, or about 65%, or about 70%, or about 75%, or about 80%. In a further example, the protein neutralises binding of a SARS-COV-2 S protein to ACE2 by about 80% to about 95% in a sVNT assay. For example, by about 80%, or about 85%, or about 90%, or about 95% in a sVNT assay. In one example, the protein neutralises binding of a SARS-COV-2 S protein to ACE2 by about 90% in a sVNT assay.

In one example, the protein neutralises binding of a SARS-COV-2 S protein to ACE2 in a Vero microneutralisation assay. For example, the protein neutralises binding of a SARS-COV-2 protein to ACE2 in a Vero microneutralisation assay as determined by reference to the inhibitory concentration (IC) value (e.g., the half maximal inhibitor concentration (IC₅₀)). In one example, the protein neutralises binding of a SARS-COV-2 S protein to ACE2 transfected VeroE6 cells with an IC₅₀ of at least about 5 μg/ml. In one example, the IC₅₀ is between about 10 μg/ml and 120 μg/ml. For example, the IC₅₀ is between about 5 μg/ml and about 10 μg/ml, for example about 5 μg/ml, or about 6 μg/ml, or about 7 μg/ml, or about 8 μg/ml, or about 9 μg/ml, or about 10 μg/ml. In one example, the protein neutralises binding of a SARS-COV-2 S protein to ACE2 transfected VeroE6 cells with an IC₅₀ of 5 μg/ml. In another example, the protein neutralises binding of a SARS-COV-2 S protein to ACE2 transfected VeroE6 cells with an IC₅₀ of 7 μg/ml. In a further example, the protein neutralises binding of a SARS-COV-2 S protein to ACE2 transfected VeroE6 cells with an IC₅₀ of 8 μg/ml. In one example, the IC₅₀ is between about 10 μg/ml and about 20 μg/ml, for example about 10 μg/ml, or about 12 μg/ml, or about 14 μg/ml, or about 16 μg/ml, or about 18 μg/ml, or about 20 μg/ml. In one example, the protein neutralises binding of a SARS-COV-2 S protein to ACE2 transfected VeroE6 cells with an IC₅₀ of 12 μg/ml. In another example, the protein neutralises binding of a SARS-COV-2 S protein to ACE2 transfected VeroE6 cells with an IC₅₀ of 14 μg/ml. In a further example, the protein neutralises binding of a SARS-COV-2 S protein to ACE2 transfected VeroE6 cells with an IC₅₀ of 20 μg/ml. In one example, the IC₅₀ is between about 20 μg/ml and about 50 μg/ml, for example about 20 μg/ml, or about 25 μg/ml, or about 30 μg/ml, or about 35 μg/ml, or about 40 μg/ml, or about 45 μg/ml, or about 50 μg/ml. In one example, the protein neutralises binding of a SARS-COV-2 S protein to ACE2 transfected VeroE6 cells with an IC₅₀ of 26 μg/ml. In another example, the protein neutralises binding of a SARS-COV-2 S protein to ACE2 transfected VeroE6 cells with an IC₅₀ of 28 μg/ml. In a further example, the protein neutralises binding of a SARS-COV-2 S protein to ACE2 transfected VeroE6 cells with an IC₅₀ of 32 μg/ml. In another example, the protein neutralises binding of a SARS-COV-2 S protein to ACE2 transfected VeroE6 cells with an IC₅₀ of 50 μg/ml. In one example, the IC₅₀ is between about 50 μg/ml and about 100 μg/ml, for example about 50 μg/ml, or about 60 μg/ml, or about 70 μg/ml, or about 80 μg/ml, or about 90 μg/ml, or about 100 μg/ml. In one example, the protein neutralises binding of a SARS-COV-2 S protein to ACE2 transfected VeroE6 cells with an IC₅₀ of 60 μg/ml. In another example, the protein neutralises binding of a SARS-COV-2 S protein to ACE2 transfected VeroE6 cells with an IC₅₀ of 76 μg/ml. In a further example, the protein neutralises binding of a SARS-COV-2 S protein to ACE2 transfected VeroE6 cells with an IC₅₀ of 78 μg/ml. In another example, the protein neutralises binding of a SARS-COV-2 S protein to ACE2 transfected VeroE6 cells with an IC₅₀ of 96 μg/ml. In one example, the IC₅₀ is between about 100 μg/ml and about 120 μg/ml, for example about 100 μg/ml, or about 105 μg/ml, or about 110 μg/ml, or about 115 μg/ml, or about 120 μg/ml. In one example, the protein neutralises binding of a SARS-COV-2 S protein to ACE2 transfected VeroE6 cells with an IC₅₀ of 118 μg/ml.

In one example, the protein neutralises binding of a SARS-COV-2 S protein to ACE2 in a pseudovirus neutralising assay (PsV). For example, the protein neutralises binding of SARS-COV-2 S protein to ACE2 expressing cells (e.g., HeLa-ACE or HEK-293T). In one example, the protein neutralises binding of a SARS-COV-2 S protein to ACE2 in a PsV assay as determined by reference to the relative luminescence units (RLU). In one example, the RLU is expressed as the IC₅₀. For example, the concentration of protein required for a 50% reduction of RLU compared to the RLU in a control sample (i.e., where the protein is absent or a known anti-CoV-2 antibody is present). In one example, the IC₅₀ is between about 1 μg/ml and about 10 μg/ml. For example, the IC₅₀ is about 1 μg/ml, or about 2 μg/ml, or about 3 μg/ml, or about 4 μg/ml, or about 5 μg/ml, or about 6 μg/ml, or about 7 μg/ml, or about 8 μg/ml, or about 9 μg/ml, or about 10 μg/ml. In one example, the IC₅₀ is about 1.5 μg/ml. In another example, the IC₅₀ is about 4.5 μg/ml. In a further example, the IC₅₀ is about 10 μg/ml. In another example, the RLU is expressed as the area under the neutralisation curve (AUC). For example, AUC is between about 15,000 and 65,000. For example, the AUC is about 15,000. In another example, the AUC is about 55,000. In a further example, the AUC is about 55,000.

In one example, the protein comprises an antibody variable region which:
(i) binds specifically to a conformational epitope in the SARS-COV-2 S protein RBD, the RBD comprising amino acid residues 5, 6, 17, 18 and 19 of a sequence set forth in SEQ ID NO: 51;
(ii) binds to a mutant S protein, wherein the mutant S protein comprises:
   (a) D to G mutation at residue corresponding to nucleotide 614 of SEQ ID NO: 50; and/or
   (b) N to Y mutation at residue corresponding to nucleotide 501 of SEQ ID NO: 50; and/or
   (c) S to N mutation at residue corresponding to nucleotide 477 of SEQ ID NO: 50; and/or
   (d) G to R mutation at residue corresponding to nucleotide 485 of SEQ ID NO: 50; and/or
(iii) binds specifically to the same epitope in the SARS-COV-2 S protein RBD as that bound by any one of the following antibodies:
   (a) an antibody comprising a V_{H} comprising a sequence set forth in SEQ ID NO: 1 and a V_{L} comprising a sequence set forth in SEQ ID NO: 2;
   (b) an antibody comprising a V_{H} comprising a sequence set forth in SEQ ID NO: 3 and a V_{L} comprising a sequence set forth in SEQ ID NO: 4;
   (c) an antibody comprising a V_{H} comprising a sequence set forth in SEQ ID NO: 5 and a V_{L} comprising a sequence set forth in SEQ ID NO: 6;
   (d) an antibody comprising a V_{H} comprising a sequence set forth in SEQ ID NO: 7 and a V_{L} comprising a sequence set forth in SEQ ID NO: 8; and
   (e) an antibody comprising a V_{H} comprising a sequence set forth in SEQ ID NO: 9 and a V_{L} comprising a sequence set forth in SEQ ID NO: 10.

In one example, the protein comprises an antibody variable region which binds specifically to a conformational epitope in the SARS-COV-2 S protein RBD, the RBD comprising amino acid residues 5, 6, 17, 18 and 19 of a sequence set forth in SEQ ID NO: 51.

In one example, the protein comprises an antibody variable region which binds specifically to a conformational epitope in the SARS-COV-2 S protein RBD, the RBD comprising amino acid residues 475, 476, 487, 488 and 489 of a sequence set forth in SEQ ID NO: 50.

In one example, the protein comprises an antibody variable region which binds to a mutant S protein.

In one example, a mutant S protein comprises a mutation in the receptor binding domain. For example, the mutation is selected from the group consisting of R346K, K417N, K417T, S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H and Y508H. In one example, a mutant S protein comprises a mutation in the receptor binding domain selected from the group consisting of R346K, K417N, K417T, N439K, N439L, L452R, S477N, T478I, V483A, E484D, E484K and N501Y.

In one example, a mutant S protein comprises a mutation selected from the group consisting of T951, Y144S, Y145N, P337S, F338L, F338C, G339D, E340K, V341I, A344S, T345S, R346K, A348S, A348T, W353R, N354D, N354K, N354S, S359N, D364Y, V367F, S373L, V382L, P384L, P384S, T385A, T393P, V395I, F400C, R403K, R403S, D405V, R408I, Q414E, Q414K, Q414P, Q414R, T415S, K417N, K417T, K417R, 1418V, Y421S, Y423C, Y423F, Y423S, D427Y, S438F, N439K, N440K, L441I, K444R, V445A, V445I, G446V, G446S, N450K, L452R, L452P, L455F, K458N, N460T, D467V, I468F, I468T, I468V, E4710, 1472V, A475V, G476S, S477G, S477I, S477N, S477R, T478I, T478K, P479L, P479S, N481D, N481H, V483F, V483A, E484D, E484K, E484K, E4840, G485S, Y489H, Y489D, Y489F, Y489C, Y489N, F490L, F490S, P491R, Q493L, S494P, Y495N, T500N, N501S, N501Y, Y505H, Y508H, R509K, V510L, V511E, V512L, L518I, H5190, A520S, A520V, P521R, P521S, A522P, A522S, D614G, P681H. and D950N.

In one example, the mutant S protein: (i) lacks a furin cleavage site at the S1/S2 boundary and comprises RRAR to QQAA mutations at residues corresponding to nucleotides 682-685 of SEQ ID NO: 50; and/or (ii) lacks a furin cleavage site at the S2′ site; and/or (iii) comprises D to G mutation at residue corresponding to nucleotide 614 of SEQ ID NO: 50; and/or (iv) comprises insertion of two proline residues between residues corresponding to nucleotides 986 and 987 of SEQ ID NO: 50.

In one example, the S protein lacks a furin cleavage site at the S1/S2 boundary and comprises RRAR to QQAA mutations at residues corresponding to nucleotides 682-685 of SEQ ID NO: 50.

In one example, the S protein lacks a furin cleavage site at the S2′ site.

In one example, the S protein comprises D to G mutation at residue corresponding to nucleotide 614 of SEQ ID NO: 50.

In one example, the S protein comprises insertion of two proline residues between residues corresponding to nucleotides 986 and 987 of SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) lacks a furin cleavage site at the S2′ site. For example, the mutant S protein is encoded by a sequence set forth in SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) comprises D to G mutation at residue corresponding to nucleotide 614 of SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) comprises insertion of two proline residues between residues corresponding to nucleotides 986 and 987 of SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) lacks a furin cleavage site at the S2′ site; and (iii) comprises D to G mutation at residue corresponding to nucleotide 614 of SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) lacks a furin cleavage site at the S2′ site; and (iii) comprises insertion of two proline residues between residues corresponding to nucleotides 986 and 987 of SEQ ID NO: 50.

In one example, the S protein (i) lacks a furin cleavage site at the S2′ site; and (ii) comprises D to G mutation at residue corresponding to nucleotide 614 of SEQ ID NO: 50.

In one example, the S protein (i) lacks a furin cleavage site at the S2′ site; and (ii) comprises insertion of two proline residues between residues corresponding to nucleotides 986 and 987 of SEQ ID NO: 50.

In one example, the S protein (i) lacks a furin cleavage site at the S2′ site; and (ii) comprises D to G mutation at residue corresponding to nucleotide 614 of SEQ ID NO: 50; and (iii) comprises insertion of two proline residues between residues corresponding to nucleotides 986 and 987 of SEQ ID NO: 50.

In one example, the S protein (i) comprises D to G mutation at residue corresponding to nucleotide 614 of SEQ ID NO: 50; and (ii) comprises insertion of two proline residues between residues corresponding to nucleotides 986 and 987 of SEQ ID NO: 50.

In one example, the S protein (i) lacks a furin cleavage site at the S1/S2 boundary and comprises RRAR to QQAA mutations at residues corresponding to nucleotides 682-685 of SEQ ID NO: 50; and (ii) lacks a furin cleavage site at the S2′ site; and (iii) comprises D to G mutation at residue corresponding to nucleotide 614 of SEQ ID NO: 50; and (iv) comprises insertion of two proline residues between residues corresponding to nucleotides 986 and 987 of SEQ ID NO: 50.

In one example, the mutant S protein comprises (i) a N to Y mutation at residue corresponding to nucleotide 501 of SEQ ID NO: 50; and/or (ii) deletion of two residues corresponding to nucleotides 69 and 70 of SEQ ID NO: 50; and/or (iii) P to H mutation at residue corresponding to nucleotide 681 of SEQ ID NO: 50.

In one example, the mutant S protein comprises a N to Y mutation at a residue corresponding to nucleotide 501 of SEQ ID NO: 50, and deletion of two residues corresponding to nucleotides 69 and 70 of SEQ ID NO: 50, and a P to H mutation at a residue corresponding to nucleotide 681 of SEQ ID NO: 50.

In one example, the mutant S protein comprises a P to H mutation at a residue corresponding to nucleotide 681 of SEQ ID NO: 50.

In one example, the mutant S protein comprises (i) a K to N mutation at a residue corresponding to nucleotide 417 of SEQ ID NO: 50; and/or (ii) E to K mutation at residue corresponding to nucleotide 484 of SEQ ID NO: 50; and/or (iii) a N to Y mutation at a residue corresponding to nucleotide 501 of SEQ ID NO: 50.

In one example, the mutant S protein comprises a K to N mutation at a residue corresponding to nucleotide 417 of SEQ ID NO: 50.

In one example, the mutant S protein comprises a E to K mutation at a residue corresponding to nucleotide 484 of SEQ ID NO: 50.

In one example, the mutant S protein comprises a K to N mutation at a residue corresponding to nucleotide 417 of SEQ ID NO: 50, and a E to K mutation at a residue corresponding to nucleotide 484 of SEQ ID NO: 50, and a N to Y mutation at residue corresponding to nucleotide 501 of SEQ ID NO: 50.

In one example, the mutant S protein comprises (i) a K to T mutation at a residue corresponding to nucleotide 417 of SEQ ID NO: 50; and/or (ii) a E to K mutation at a residue corresponding to nucleotide 484 of SEQ ID NO: 50; and/or (iii) a N to Y mutation at a residue corresponding to nucleotide 501 of SEQ ID NO: 50.

In one example, the mutant S protein comprises a K to T mutation at a residue corresponding to nucleotide 417 of SEQ ID NO: 50.

In one example, the mutant S protein comprises a K to T mutation at a residue corresponding to nucleotide 417 of SEQ ID NO: 50, and a E to K mutation at a residue corresponding to nucleotide 484 of SEQ ID NO: 50, and a N to Y mutation at a residue corresponding to nucleotide 501 of SEQ ID NO: 50.

In one example, the mutant S protein comprises (i) a T to I mutation at a residue corresponding to nucleotide 95 of SEQ ID NO: 50; and/or (ii) a Y to S mutation at a residue corresponding to nucleotide 144 of SEQ ID NO: 50; and/or (iii) a Y to N mutation at a residue corresponding to nucleotide 145 of SEQ ID NO: 50; and/or (iv) a R to K mutation at a residue corresponding to nucleotide 346 of SEQ ID NO: 50; and/or (v) an E to K mutation at a residue corresponding to nucleotide 484 of SEQ ID NO: 50; and/or (vi) a N to Y mutation at a residue corresponding to nucleotide 501 of SEQ ID NO: 50; and/or (vii) a D to G mutation at a residue corresponding to nucleotide 614 of SEQ ID NO: 50; and/or (viii) a P to H mutation at a residue corresponding to nucleotide 681 of SEQ ID NO: 50; and/or (ix) a D to N mutation at a residue corresponding to nucleotide 950 of SEQ ID NO: 50.

In one example, the mutant S protein comprises a T to I mutation at a residue corresponding to nucleotide 95 of SEQ ID NO: 50.

In one example, the mutant S protein comprises a Y to S mutation at a residue corresponding to nucleotide 144 of SEQ ID NO: 50.

In one example, the mutant S protein comprises a Y to N mutation at a residue corresponding to nucleotide 145 of SEQ ID NO: 50.

In one example, the mutant S protein comprises a R to K mutation at a residue corresponding to nucleotide 346 of SEQ ID NO: 50.

In one example, the mutant S protein comprises a D to N mutation at a residue corresponding to nucleotide 950 of SEQ ID NO: 50.

In one example, the mutant S protein comprises (i) a T to I mutation at a residue corresponding to nucleotide 95 of SEQ ID NO: 50; and (ii) a Y to S mutation at a residue corresponding to nucleotide 144 of SEQ ID NO: 50; and (iii) a Y to N mutation at a residue corresponding to nucleotide 145 of SEQ ID NO: 50; and (iv) a R to K mutation at a residue corresponding to nucleotide 346 of SEQ ID NO: 50; and (v) an E to K mutation at a residue corresponding to nucleotide 484 of SEQ ID NO: 50; and (vi) a N to Y mutation at a residue corresponding to nucleotide 501 of SEQ ID NO: 50; and (vii) a D to G mutation at a residue corresponding to nucleotide 614 of SEQ ID NO: 50; (viii) a P to H mutation at a residue corresponding to nucleotide 681 of SEQ ID NO: 50; (ix) a D to N mutation at a residue corresponding to nucleotide 950 of SEQ ID NO: 50.

In one example, the mutant S protein comprises (i) a T to K mutation at a residue corresponding to nucleotide 478 of SEQ ID NO: 50; and/or (ii) a P to R mutation at a residue corresponding to nucleotide 681 of SEQ ID NO: 50; and/or (iii) a L to R mutation at a residue corresponding to nucleotide 452 of SEQ ID NO: 50.

In one example, the mutant S protein comprises a T to K mutation at a residue corresponding to nucleotide 478 of SEQ ID NO: 50.

In one example, the mutant S protein comprises a P to R mutation at a residue corresponding to nucleotide 681 of SEQ ID NO: 50.

In one example, the mutant S protein comprises a L to R mutation at a residue corresponding to nucleotide 452 of SEQ ID NO: 50.

In one example, the mutant S protein comprises (i) a T to K mutation at a residue corresponding to nucleotide 478 of SEQ ID NO: 50; and (ii) a P to R mutation at a residue corresponding to nucleotide 681 of SEQ ID NO: 50; and (iii) a L to R mutation at a residue corresponding to nucleotide 452 of SEQ ID NO: 50.

In one example, the protein comprises an antibody variable region which binds to a mutant S protein, wherein the mutant S protein comprises:
(a) D to G mutation at residue corresponding to nucleotide 614 of SEQ ID NO: 50; and/or
(b) N to Y mutation at residue corresponding to nucleotide 501 of SEQ ID NO: 50; and/or
(c) S to N mutation at residue corresponding to nucleotide 477 of SEQ ID NO: 50; and/or
(d) G to R mutation at residue corresponding to nucleotide 485 of SEQ ID NO: 50.

In one example, the protein comprises an antibody variable region which binds to a mutant S protein, wherein the mutant S protein comprises D to G mutation at residue corresponding to nucleotide 614 of SEQ ID NO: 50.

In one example, the protein comprises an antibody variable region which binds to a mutant S protein, wherein the mutant S protein comprises N to Y mutation at residue corresponding to nucleotide 501 of SEQ ID NO: 50.

In one example, the protein comprises an antibody variable region which binds to a mutant S protein, wherein the mutant S protein comprises S to N mutation at residue corresponding to nucleotide 477 of SEQ ID NO: 50.

In one example, the protein comprises an antibody variable region which binds to a mutant S protein, wherein the mutant S protein comprises G to R mutation at residue corresponding to nucleotide 485 of SEQ ID NO: 50.

In one example, the protein comprises an antibody variable region which binds to a mutant S protein, wherein the mutant S protein comprises S to N mutation at residue corresponding to nucleotide 477 of SEQ ID NO: 50 and D to G mutation at residue corresponding to nucleotide 614 of SEQ ID NO: 50.

In one example, the protein comprises an antibody variable region which binds to a mutant S protein, wherein the mutant S protein comprises G to R mutation at residue corresponding to nucleotide 485 of SEQ ID NO: 50 and D to G mutation at residue corresponding to nucleotide 614 of SEQ ID NO: 50.

In one example, the protein comprises an antibody variable region which binds to a mutant S protein, wherein the mutant S protein comprises N to Y mutation at residue corresponding to nucleotide 501 of SEQ ID NO: 50 and D to G mutation at residue corresponding to nucleotide 614 of SEQ ID NO: 50.

In one example, the protein comprises an antibody variable region which binds specifically to the same epitope in the SARS-COV-2 S protein RBD as that bound by an antibody comprising a V_{H} comprising a sequence set forth in SEQ ID NO: 1 and a V_{L} comprising a sequence set forth in SEQ ID NO: 2.

In one example, the protein comprises an antibody variable region which binds specifically to the same epitope in the SARS-COV-2 S protein RBD as that bound by an antibody comprising a V_{H} comprising a sequence set forth in SEQ ID NO: 3 and a V_{L} comprising a sequence set forth in SEQ ID NO: 4.

In one example, the protein comprises an antibody variable region which binds specifically to the same epitope in the SARS-COV-2 S protein RBD as that bound by an antibody comprising a V_{H} comprising a sequence set forth in SEQ ID NO: 5 and a V_{L} comprising a sequence set forth in SEQ ID NO: 6.

In one example, the protein comprises an antibody variable region which binds specifically to the same epitope in the SARS-COV-2 S protein RBD as that bound by an antibody comprising a V_{H} comprising a sequence set forth in SEQ ID NO: 7 and a V_{L} comprising a sequence set forth in SEQ ID NO: 8.

In one example, the protein comprises an antibody variable region which binds specifically to the same epitope in the SARS-COV-2 S protein RBD as that bound by an antibody comprising a V_{H} comprising a sequence set forth in SEQ ID NO: 9 and a V_{L} comprising a sequence set forth in SEQ ID NO: 10.

In one example, the protein comprises an antibody variable region which cross-reacts with a peptide comprising a sequence set forth in SEQ ID NO: 51. In one example, the protein comprises an antibody variable region which cross-reacts with a peptide comprising amino acid residues 5, 6, 17, 18 and 19 of a sequence set forth in SEQ ID NO: 51. An exemplary protein which cross-reacts with a peptide comprising a sequence set forth in SEQ ID NO: 51 is anti-SARS-COV-2 monoclonal antibody 30B8. An exemplary protein which does not cross-react with a peptide comprising a sequence set forth in SEQ ID NO: 51 is anti-SARS-COV-2 antibody AS35. A further exemplary protein which does not cross-react with a peptide comprising a sequence set forth in SEQ ID NO: 51 is anti-SARS-COV-2 antibody B38.

Methods of determining cross-reactivity will be apparent to the skilled person and/or are described herein.

In one example, the protein comprises a fragment variable (Fv).

In one example, the protein is selected from the group consisting of:
(i) a single chain fragment variable (Fv) fragment (scFv);
(ii) a dimeric scFv (di-scFv);
(iii) a diabody;
(iv) a triabody;
(v) a tetrabody;
(vi) a fragment antigen binding (Fab);
(vii) a F(ab′)₂;
(viii) a Fv;
(ix) one of (i) to (viii) linked to a constant region of an antibody, a constant fragment (Fc) or a heavy chain constant domain (C_{H})₂ and/or C_{H}3; or
(x) an antibody.

For example, the protein comprises a V_{H} and a V_{L} wherein the V_{H} and the V_{L} are in a single polypeptide chain and the protein is selected from the group consisting of:
(a) a scFv;
(b) a di-scFv; and
(c) one of (a) or (b) linked to a constant region of an antibody, a Fc or a CH2 and/or CH3.

In another example, the protein comprises a V_{H} and a V_{L}, wherein the V_{H} and the V_{L} are in separate polypeptide chains and the protein is selected from the group consisting of:
(a) a diabody;
(b) a triabody;
(c) a tetrabody;
(d) a Fab;
(e) a F(ab′)₂;
(f) a Fv;
(g) one of (a) to (f) linked to a constant region of an antibody, a Fc or a CH2 and/or CH3; and
(h) an antibody.

In one example, the protein is an antibody. Exemplary antibodies are full length and/or naked antibodies. For example, the protein is an anti-SARS-COV-2 antibody. In one example, the anti-SARS-COV-2 antibody is a monoclonal anti-SARS-COV-2 antibody.

In one example, the protein is recombinant, chimeric, CDR grafted, humanized, synhumanized, primatized, deimmunized or human.

In one example, the protein is an antibody comprising:
(i) a V_{H} comprising a sequence set forth in SEQ ID NO: 45 and a V_{L} comprising a sequence set forth in SEQ ID NO: 46 or SEQ ID NO: 6;
(ii) a V_{H} comprising three complementarity determining regions (CDRs) of a V_{H} comprising a sequence set forth in SEQ ID NO: 45 and a V_{L} comprising three CDRs of a V_{L} comprising a sequence set forth in SEQ ID NO: 46 or SEQ ID NO: 6; or
(iii) a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 36 or SEQ ID NO: 32, a CDR2 comprising a sequence set forth in SEQ ID NO: 37 or SEQ ID NO: 33, and a CDR3 comprising a sequence set forth in SEQ ID NO: 38 or SEQ ID NO: 34.

In one example, the protein is an antibody comprising a V_{H} comprising a sequence set forth in SEQ ID NO: 45 and a V_{L} comprising a sequence set forth in SEQ ID NO: 46.

In one example, the amino acid sequence of the V_{H} comprises a sequence set forth in SEQ ID NO: 45, wherein the amino acid sequence comprises Alanine (A) or Valine (V) at position 24 and/or Glycine (G) or Aspartic Acid (D) at position 26 and/or Methionine (M) or Isoleucine (I) at position 34 and/or Serine(S) or Asparagine (N) at position 55 and/or Glycine (G) or Aspartic Acid (D) at position 56 and/or Threonine (T) or Serine(S) at position 58 and/or Alanine (A) or Proline (P) at position 72 and/or Cysteine (C) or Tyrosine (Y) at position 94 and/or Glycine (G) or Serine(S) at position 98 and/or Leucine (L) or Tyrosine (Y) at position 100 and/or Alanine (A) or Serine(S) at position 106 and/or Proline (P) or Tryptophan (W) at position 107.

In one example, the amino acid sequence of the V_{L} comprises a sequence set forth in SEQ ID NO: 46, wherein the amino acid sequence comprises Serine(S) or Arginine (R) at position 26 and/or Isoleucine (I) or Valine (V) at position 45 and/or Arginine (R) or Lysine (K) at position 102.

In one example, the protein is an antibody comprising a V_{H} comprising a sequence set forth in SEQ ID NO: 45 and a V_{L} comprising a sequence set forth in SEQ ID NO: 46. For example, the amino acid sequence of the V_{H} comprises a sequence set forth in SEQ ID NO: 45, wherein the amino acid sequence comprises Valine (V) at position 24, Glycine (G) at position 26, Methionine (M) at position 34, Asparagine (N) at position 55, Glycine (G) at position 56, Threonine (T) at position 58, Alanine (A) at position 72, Tyrosine (Y) at position 94, Serine(S) at position 98, Tyrosine (Y) at position 100, Serine(S) at position 106, and Tryptophan (W) at position 107; and the amino acid sequence of the V_{L} comprises a sequence set forth in SEQ ID NO: 46, wherein the amino acid sequence comprises Serine(S) at position 26, Isoleucine (I) at position 45, and Arginine (R) at position 102.

In one example, the protein is an antibody comprising a V_{H} comprising a sequence set forth in SEQ ID NO: 45 and a V_{L} comprising a sequence set forth in SEQ ID NO: 46. For example, the amino acid sequence of the V_{H} comprises a sequence set forth in SEQ ID NO: 45, wherein the amino acid sequence comprises Alanine (A) at position 24, Aspartic Acid (D) at position 26, Methionine (M) at position 34, Serine(S) at position 55, Aspartic Acid (D) at position 56, Serine(S) at position 58, Proline (P) at position 72, Tyrosine (Y) at position 94, Serine(S) at position 98, Tyrosine (Y) at position 100, Serine(S) at position 106, and Tryptophan (W) at position 107; and the amino acid sequence of the V_{L} comprises a sequence set forth in SEQ ID NO: 46, wherein the amino acid sequence comprises Arginine (R) at position 26, and Valine (V) at position 45, Lysine (K) at position 102.

In one example, the protein is an antibody comprising a V_{H} comprising a sequence set forth in SEQ ID NO: 45 and a V_{L} comprising a sequence set forth in SEQ ID NO: 6. For example, the amino acid sequence of the V_{H} comprises a sequence set forth in SEQ ID NO: 45, wherein the amino acid sequence comprises Alanine (A) at position 24, Glycine (G) at position 26, Isoleucine (I) at position 34, Asparagine (N) at position 55, Glycine (G) at position 56, Threonine (T) at position 58, Alanine (A) at position 72, Cysteine (C) at position 94, Glycine (G) at position 98, Leucine (L) at position 100, Alanine (A) at position 106, and Proline (P) at position 107.

In one example, the protein is an antibody comprising a V_{H} comprising three CDRs of a V_{H} comprising a sequence set forth in SEQ ID NO: 45 and a V_{L} comprising three CDRs of a V_{L} comprising a sequence set forth in SEQ ID NO: 46 or SEQ ID NO: 6.

In one example, the protein is an antibody comprising a V_{H} comprising three CDRs of a V_{H} comprising a sequence set forth in SEQ ID NO: 45 and a V_{L} comprising three CDRs of a V_{L} comprising a sequence set forth in SEQ ID NO: 46. For example, the amino acid sequence of the V_{H} comprises a sequence set forth in SEQ ID NO: 45, wherein the amino acid sequence comprises Valine (V) at position 24, Glycine (G) at position 26, Methionine (M) at position 34, Asparagine (N) at position 55, Glycine (G) at position 56, Threonine (T) at position 58, Alanine (A) at position 72, Tyrosine (Y) at position 94, Serine(S) at position 98, Tyrosine (Y) at position 100, Serine(S) at position 106, and Tryptophan (W) at position 107; and the amino acid sequence of the V_{L} comprises a sequence set forth in SEQ ID NO: 46, wherein the amino acid sequence comprises Serine(S) at position 26, Isoleucine (I) at position 45, and Arginine (R) at position 102.

In one example, the protein is an antibody comprising a V_{H} comprising three CDRs of a V_{H} comprising a sequence set forth in SEQ ID NO: 45 and a V_{L} comprising three CDRs of a V_{L} comprising a sequence set forth in SEQ ID NO: 46. For example, the amino acid sequence of the V_{H} comprises a sequence set forth in SEQ ID NO: 45, wherein the amino acid sequence comprises Alanine (A) at position 24, Aspartic Acid (D) at position 26, Methionine (M) at position 34, Serine(S) at position 55, Aspartic Acid (D) at position 56, Serine(S) at position 58, Proline (P) at position 72, Tyrosine (Y) at position 94, Serine(S) at position 98, Tyrosine (Y) at position 100, Serine(S) at position 106, and Tryptophan (W) at position 107; and the amino acid sequence of the V_{L} comprises a sequence set forth in SEQ ID NO: 46, wherein the amino acid sequence comprises Arginine (R) at position 26, and Valine (V) at position 45, Lysine (K) at position 102.

In one example, the protein is an antibody comprising a V_{H} comprising three CDRs of a V_{H} comprising a sequence set forth in SEQ ID NO: 45 and a V_{L} comprising three CDRs of a V_{L} comprising a sequence set forth in SEQ ID NO: 6. For example, the amino acid sequence of the V_{H} comprises a sequence set forth in SEQ ID NO: 45, wherein the amino acid sequence comprises Alanine (A) at position 24, Glycine (G) at position 26, Isoleucine (I) at position 34, Asparagine (N) at position 55, Glycine (G) at position 56, Threonine (T) at position 58, Alanine (A) at position 72, Cysteine (C) at position 94, Glycine (G) at position 98, Leucine (L) at position 100, Alanine (A) at position 106, and Proline (P) at position 107.

In one example, the protein is an antibody comprising a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 36 or SEQ ID NO: 32, a CDR2 comprising a sequence set forth in SEQ ID NO: 37 or SEQ ID NO: 33, and a CDR3 comprising a sequence set forth in SEQ ID NO: 38 or SEQ ID NO: 34.

In one example, the amino acid sequence of the V_{H} CDR1 comprises a sequence set forth in SEQ ID NO: 47, wherein the amino acid sequence comprises Glycine (G) or Aspartic Acid (D) at position 1.

In one example, the amino acid sequence of the V_{H} CDR2 comprises a sequence set forth in SEQ ID NO: 48, wherein the amino acid sequence comprises Serine(S) or Asparagine (N) at position 5 and/or Glycine (G) or Aspartic Acid (D) at position 6 and/or Threonine (T) or Serine(S) at position 8.

In one example, the amino acid sequence of the V_{H} CDR3 comprises a sequence set forth in SEQ ID NO: 49, wherein the amino acid sequence comprises Glycine (G) or Serine(S) at position 2 and/or Lysine (L) or Tyrosine (Y) at position 4 and/or Alanine (A) or Serine(S) at position 10 and/or Proline (P) or Tryptophan (W) at position 11.

In one example, the protein is an antibody comprising a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 36, a CDR2 comprising a sequence set forth in SEQ ID NO: 37, and a CDR3 comprising a sequence set forth in SEQ ID NO: 38. For example, the amino acid sequence of the V_{H} CDR1 comprises a sequence set forth in SEQ ID NO: 47, wherein the amino acid sequence comprises Glycine (G) at position 1; the V_{H}CDR2 comprises a sequence set forth in SEQ ID NO: 48, wherein the amino acid sequence comprises Asparagine (N) at position 5, Glycine (G) at position 6, Threonine (T) at position 8; and the V_{H} CDR3 comprises a sequence set forth in SEQ ID NO: 49, wherein the amino acid sequence comprises Serine(S) at position 2, Tyrosine (Y) at position 4, Serine(S) at position 10, Tryptophan (W) at position 11.

In one example, the protein is an antibody comprising a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 36, a CDR2 comprising a sequence set forth in SEQ ID NO: 37, and a CDR3 comprising a sequence set forth in SEQ ID NO: 38. For example, the amino acid sequence of the V_{H} CDR1 comprises a sequence set forth in SEQ ID NO: 47, wherein the amino acid sequence comprises Aspartic Acid (D) at position 1; the V_{H} CDR2 comprises a sequence set forth in SEQ ID NO: 48, wherein the amino acid sequence comprises Serine(S) at position 5, Aspartic Acid (D) at position 6, Serine(S) at position 8; and the V_{H} CDR3 comprises a sequence set forth in SEQ ID NO: 49, wherein the amino acid sequence comprises Serine(S) at position 2, Tyrosine (Y) at position 4, Serine(S) at position 10, Tryptophan (W) at position 11.

In one example, the protein is an antibody comprising a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 32, a CDR2 comprising a sequence set forth in SEQ ID NO: 33, and a CDR3 comprising a sequence set forth in SEQ ID NO: 34. For example, the amino acid sequence of the V_{H} CDR1 comprises a sequence set forth in SEQ ID NO: 47, wherein the amino acid sequence comprises Glycine (G) at position 1; the V_{H} CDR2 comprises a sequence set forth in SEQ ID NO: 48, wherein the amino acid sequence comprises Asparagine (N) at position 5, Glycine (G) at position 6, Threonine (T) at position 8; and the V_{H} CDR3 comprises a sequence set forth in SEQ ID NO: 49, wherein the amino acid sequence comprises Glycine (G) at position 2, Lysine (L), Alanine (A) at position 10, Proline (P) at position 11.

In one example, the protein is an antibody comprising:
(i) a heavy chain variable region (V_{H}) comprising:
   (a) a complementarity determining region (CDR) 1 comprising a sequence set forth in:
      a. amino acids 26 to 34 of SEQ ID NO: 1; or
      b. amino acids 26 to 33 of SEQ ID NO: 3; or
      c. amino acids 26 to 33 of SEQ ID NO: 5; or
      d. amino acids 26 to 33 of SEQ ID NO: 7; or
      e. amino acids 26 to 33 of SEQ ID NO: 9; and
   (b) a CDR2 comprising a sequence set forth in:
      a. amino acids 52 to 58 of SEQ ID NO: 1; or
      b. amino acids 51 to 58 of SEQ ID NO: 3; or
      c. amino acids 51 to 58 of SEQ ID NO: 5; or
      d. amino acids 26 to 33 of SEQ ID NO: 7; or
      e. amino acids 51 to 58 of SEQ ID NO: 9; and
   (c) a CDR3 comprising a sequence set forth in:
      a. amino acids 97 to 108 of SEQ ID NO: 1; or
      b. amino acids 97 to 110 of SEQ ID NO: 3; or
      c. amino acids 97 to 110 of SEQ ID NO: 5; or
      d. amino acids 97 to 110 of SEQ ID NO: 7; or
      e. amino acids 97 to 106 of SEQ ID NO: 9; and
(ii) a light chain variable region (V_{L}) comprising:
   (a) a CDR1 comprising a sequence set forth in:
      a. amino acids 27 to 38 of SEQ ID NO: 2; or
      b. amino acids 27 to 31 of SEQ ID NO: 4; or
      c. amino acids 27 to 32 of SEQ ID NO: 6; or
      d. amino acids 27 to 31 of SEQ ID NO: 8; or
      e. amino acids 27 to 37 of SEQ ID NO: 10; and
   (b) a CDR2 comprising a sequence set forth in:
      a. amino acids 56 to 58 of SEQ ID NO: 2; or
      b. amino acids 49 to 51 of SEQ ID NO: 4; or
      c. amino acids 50 to 52 of SEQ ID NO: 6; or
      d. amino acids 49 to 51 of SEQ ID NO: 8; or
      e. amino acids 55 to 57 of SEQ ID NO: 10; and
   (c) a CDR3 comprising a sequence set forth in:
      a. amino acids 95 to 103 of SEQ ID NO: 2; or
      b. amino acids 88 to 96 of SEQ ID NO: 4; or
      c. amino acids 89 to 97 of SEQ ID NO: 6; or
      d. amino acids 88 to 96 of SEQ ID NO: 8; or
      e. amino acids 94 to 102 of SEQ ID NO: 10.
In one example, the protein is an antibody comprising:
   (i) a heavy chain variable region (V_{H}) comprising:
      (a) a complementarity determining region (CDR) 1 comprising a sequence set forth in amino acids 26 to 34 of SEQ ID NO: 1; and
      (b) a CDR2 comprising a sequence set forth in amino acids 52 to 58 of SEQ ID NO: 1; and
      (c) a CDR3 comprising a sequence set forth in amino acids 97 to 108 of SEQ ID NO: 1; and
   (ii) a light chain variable region (V_{L}) comprising:
      (a) a CDR1 comprising a sequence set forth in amino acids 27 to 38 of SEQ ID NO: 2; and
      (b) a CDR2 comprising a sequence set forth in amino acids 56 to 58 of SEQ ID NO: 2; and
      (c) a CDR3 comprising a sequence set forth in amino acids 95 to 103 of SEQ ID NO: 2.

In one example, the protein is an antibody comprising:
(i) a heavy chain variable region (V_{H}) comprising:
   (a) a complementarity determining region (CDR) 1 comprising a sequence set forth in amino acids 26 to 33 of SEQ ID NO: 3; and
   (b) a CDR2 comprising a sequence set forth in amino acids 51 to 58 of SEQ ID NO: 3; and
   (c) a CDR3 comprising a sequence set forth in amino acids 97 to 110 of SEQ ID NO: 3; and
(ii) a light chain variable region (V_{L}) comprising:
   (a) a CDR1 comprising a sequence set forth in amino acids 27 to 31 of SEQ ID NO: 4; and
   (b) a CDR2 comprising a sequence set forth in amino acids 49 to 51 of SEQ ID NO: 4; and
   (c) a CDR3 comprising a sequence set forth in amino acids 88 to 96 of SEQ ID NO: 4.

In one example, the protein is an antibody comprising:
(i) a heavy chain variable region (V_{H}) comprising:
   (a) a complementarity determining region (CDR) 1 comprising a sequence set forth in amino acids 26 to 33 of SEQ ID NO: 5; and
   (b) a CDR2 comprising a sequence set forth in amino acids 51 to 58 of SEQ ID NO: 5; and
   (c) a CDR3 comprising a sequence set forth in amino acids 97 to 110 of SEQ ID NO: 5; and
(ii) a light chain variable region (V_{L}) comprising:
   (a) a CDR1 comprising a sequence set forth in amino acids 27 to 32 of SEQ ID NO: 6; and
   (b) a CDR2 comprising a sequence set forth in amino acids 50 to 52 of SEQ ID NO: 6; and
   (c) a CDR3 comprising a sequence set forth in amino acids 89 to 97 of SEQ ID NO: 6.

In one example, the protein is an antibody comprising:
(i) a heavy chain variable region (V_{H}) comprising:
   (a) a complementarity determining region (CDR) 1 comprising a sequence set forth in amino acids 26 to 33 of SEQ ID NO: 7; and
   (b) a CDR2 comprising a sequence set forth in amino acids 26 to 33 of SEQ ID NO: 7; and
   (c) a CDR3 comprising a sequence set forth in amino acids 97 to 110 of SEQ ID NO: 7; and
(ii) a light chain variable region (V_{L}) comprising:
   (a) a CDR1 comprising a sequence set forth in amino acids 27 to 31 of SEQ ID NO: 8; and
   (b) a CDR2 comprising a sequence set forth in amino acids 49 to 51 of SEQ ID NO: 8; and
   (c) a CDR3 comprising a sequence set forth in amino acids 88 to 96 of SEQ ID NO: 8.

In one example, the protein is an antibody comprising:
(i) a heavy chain variable region (V_{H}) comprising:
   (a) a complementarity determining region (CDR) 1 comprising a sequence set forth in amino acids 26 to 33 of SEQ ID NO: 9; and
   (b) a CDR2 comprising a sequence set forth in amino acids 51 to 58 of SEQ ID NO: 9; and
   (c) a CDR3 comprising a sequence set forth in amino acids 97 to 106 of SEQ ID NO: 9; and
(ii) a light chain variable region (V_{L}) comprising:
   (a) a CDR1 comprising a sequence set forth in amino acids 27 to 37 of SEQ ID NO: 10; and
   (b) a CDR2 comprising a sequence set forth in amino acids 55 to 57 of SEQ ID NO: 10; and
   (c) a CDR3 comprising a sequence set forth in amino acids 94 to 102 of SEQ ID NO: 10.

In one example, the protein is an antibody comprising:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8; or
(v) a V_{H} comprising a sequence set forth in SEQ ID NO: 9 and a V_{L} comprising a sequence set forth in SEQ ID NO: 10.

In one example, the protein is an antibody comprising a V_{H} comprising an amino acid sequence set forth in SEQ ID NO: 1 and a V_{L} comprising an amino acid sequence set forth in any one of SEQ ID NO: 2.

In one example, the protein is an antibody comprising a V_{H} comprising an amino acid sequence set forth in SEQ ID NO: 3 and a V_{L} comprising an amino acid sequence set forth in any one of SEQ ID NO: 4.

In one example, the protein is an antibody comprising a V_{H} comprising an amino acid sequence set forth in SEQ ID NO: 5 and a V_{L} comprising an amino acid sequence set forth in any one of SEQ ID NO: 6.

In one example, the protein is an antibody comprising a V_{H} comprising an amino acid sequence set forth in SEQ ID NO: 7 and a V_{L} comprising an amino acid sequence set forth in any one of SEQ ID NO: 8.

In one example, the protein is an antibody comprising a V_{H} comprising an amino acid sequence set forth in SEQ ID NO: 9 and a V_{L} comprising an amino acid sequence set forth in any one of SEQ ID NO: 10.

In one example, the protein is an antibody comprising:
(i) a V_{H} comprising:
   (a) a CDR1 comprising a sequence set forth in SEQ ID NO: 21;
   (b) a CDR2 comprising a sequence set forth in SEQ ID NO: 22; and
   (c) a CDR3 comprising a sequence set forth in SEQ ID NO: 23; and
a V_{L} comprising:
   (a) a CDR1 comprising a sequence set forth in SEQ ID NO: 24;
   (b) a CDR2 comprising a sequence set forth in SEQ ID NO: 25; and
   (c) a CDR3 comprising a sequence set forth in SEQ ID NO: 26; or
(ii) a V_{H} comprising:
   (a) a CDR1 comprising a sequence set forth in SEQ ID NO: 29;
   (b) a CDR2 comprising a sequence set forth in SEQ ID NO: 30; and
   (c) a CDR3 comprising a sequence set forth in SEQ ID NO: 35; and
a V_{L} comprising:
   (a) a CDR1 comprising a sequence set forth in SEQ ID NO: 36;
   (b) a CDR2 comprising a sequence set forth in SEQ ID NO: 37; and
   (c) a CDR3 comprising a sequence set forth in SEQ ID NO: 38; or
(iii) a V_{H} comprising:
   (a) a CDR1 comprising a sequence set forth in SEQ ID NO: 27;
   (b) a CDR2 comprising a sequence set forth in SEQ ID NO: 28; and
   (c) a CDR3 comprising a sequence set forth in SEQ ID NO: 35; and
a V_{L} comprising:
   (a) a CDR1 comprising a sequence set forth in SEQ ID NO: 36;
   (b) a CDR2 comprising a sequence set forth in SEQ ID NO: 37; and
   (c) a CDR3 comprising a sequence set forth in SEQ ID NO: 38; or.
(iv) a V_{H} comprising:
   (a) a CDR1 comprising a sequence set forth in SEQ ID NO: 29;
   (b) a CDR2 comprising a sequence set forth in SEQ ID NO: 30; and
   (c) a CDR3 comprising a sequence set forth in SEQ ID NO: 31; and
a V_{L} comprising:
   (a) a CDR1 comprising a sequence set forth in SEQ ID NO: 32;
   (b) a CDR2 comprising a sequence set forth in SEQ ID NO: 33; and
   (c) a CDR3 comprising a sequence set forth in SEQ ID NO: 34.
(v) a V_{H} comprising:
   (a) a CDR1 comprising a sequence set forth in SEQ ID NO: 39;
   (b) a CDR2 comprising a sequence set forth in SEQ ID NO: 40; and
   (c) a CDR3 comprising a sequence set forth in SEQ ID NO: 41; and
a V_{L} comprising:
   (a) a CDR1 comprising a sequence set forth in SEQ ID NO: 42;
   (b) a CDR2 comprising a sequence set forth in SEQ ID NO: 43; and
   (c) a CDR3 comprising a sequence set forth in SEQ ID NO: 44.

In one example, the protein is an antibody comprising a V_{H} comprising a CDR1 comprising a sequence set forth in SEQ ID NO: 21, a CDR2 comprising a sequence set forth in SEQ ID NO: 22, a CDR3 comprising a sequence set forth in SEQ ID NO: 23 and a V_{L} comprising a CDR1 comprising a sequence set forth in SEQ ID NO: 24, a CDR2 comprising a sequence set forth in SEQ ID NO: 25, and a CDR3 comprising a sequence set forth in SEQ ID NO: 26.

In one example, the protein is an antibody comprising a V_{H} comprising a CDR1 comprising a sequence set forth in SEQ ID NO: 29, a CDR2 comprising a sequence set forth in SEQ ID NO: 30, a CDR3 comprising a sequence set forth in SEQ ID NO: 35 and a V_{L} comprising a CDR1 comprising a sequence set forth in SEQ ID NO: 36, a CDR2 comprising a sequence set forth in SEQ ID NO: 37, and a CDR3 comprising a sequence set forth in SEQ ID NO: 38.

In one example, the protein is an antibody comprising a V_{H} comprising a CDR1 comprising a sequence set forth in SEQ ID NO: 27, a CDR2 comprising a sequence set forth in SEQ ID NO: 28, a CDR3 comprising a sequence set forth in SEQ ID NO: 35 and a V_{L} comprising a CDR1 comprising a sequence set forth in SEQ ID NO: 36, a CDR2 comprising a sequence set forth in SEQ ID NO: 37 and a CDR3 comprising a sequence set forth in SEQ ID NO: 38.

In one example, the protein is an antibody comprising a V_{H} comprising a CDR1 comprising a sequence set forth in SEQ ID NO: 29, a CDR2 comprising a sequence set forth in SEQ ID NO: 30, a CDR3 comprising a sequence set forth in SEQ ID NO: 31 and a V_{L} comprising a CDR1 comprising a sequence set forth in SEQ ID NO: 32, a CDR2 comprising a sequence set forth in SEQ ID NO: 33 and a CDR3 comprising a sequence set forth in SEQ ID NO: 34.

In one example, the protein is an antibody comprising a V_{H} comprising a CDR1 comprising a sequence set forth in SEQ ID NO: 39, a CDR2 comprising a sequence set forth in SEQ ID NO: 40, a CDR3 comprising a sequence set forth in SEQ ID NO: 41 and a V_{L} comprising a CDR1 comprising a sequence set forth in SEQ ID NO: 42, a CDR2 comprising a sequence set forth in SEQ ID NO: 43, and a CDR3 comprising a sequence set forth in SEQ ID NO: 44.

The present disclosure also provides an anti-SARS-COV-2 antibody, the antibody comprising any one of the following:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8; or
(v) a V_{H} comprising a sequence set forth in SEQ ID NO: 9 and a V_{L} comprising a sequence set forth in SEQ ID NO: 10.

In one example, the anti-SARS-COV-2 antibody comprises a V_{H} comprising a sequence set forth in SEQ ID NO: 1 and a V_{L} comprising a sequence set forth in SEQ ID NO: 2.

In one example, the anti-SARS-COV-2 antibody comprises a V_{H} comprising a sequence set forth in SEQ ID NO: 3 and a V_{L} comprising a sequence set forth in SEQ ID NO: 4.

In one example, the anti-SARS-COV-2 antibody comprises a V_{H} comprising a sequence set forth in SEQ ID NO: 5 and a V_{L} comprising a sequence set forth in SEQ ID NO: 6.

In one example, the anti-SARS-COV-2 antibody comprises a V_{H} comprising a sequence set forth in SEQ ID NO: 7 and a V_{L} comprising a sequence set forth in SEQ ID NO: 8.

In one example, the anti-SARS-COV-2 antibody comprises a V_{H} comprising a sequence set forth in SEQ ID NO: 9 and a V_{L} comprising a sequence set forth in SEQ ID NO: 10.

The present disclosure also provides an anti-SARS-COV-2 antibody comprising a V_{H} and a V_{L}, wherein the V_{H} is linked to a human heavy chain constant region and the V_{L} is linked to a human light chain constant region.

In one example, the protein or antibody is any form of the protein or antibody encoded by a nucleic acid encoding any of the foregoing proteins or antibodies.

The present disclosure provides an anti-SARS-COV-2 antibody, the antibody comprising any one of the following:
(i) a V_{H} comprising a sequence expressed from or encoded by a nucleic acid comprising SEQ ID NO: 11 and a V_{L} comprising a sequence expressed from or encoded by a nucleic acid comprising SEQ ID NO: 12;
(ii) a V_{H} comprising a sequence expressed from or encoded by a nucleic acid comprising SEQ ID NO: 13 and a V_{L} comprising a sequence expressed from or encoded by a nucleic acid comprising SEQ ID NO: 14;
(iii) a V_{H} comprising a sequence expressed from or encoded by a nucleic acid comprising SEQ ID NO: 15 and a V_{L} comprising a sequence expressed from or encoded by a nucleic acid comprising SEQ ID NO: 16;
(iv) a V_{H} comprising a sequence expressed from or encoded by a nucleic acid comprising SEQ ID NO: 17 and a V_{L} comprising a sequence expressed from or encoded by a nucleic acid comprising SEQ ID NO: 18;
(v) a V_{H} comprising a sequence expressed from or encoded by a nucleic acid comprising SEQ ID NO: 17 and a V_{L} comprising a sequence expressed from or encoded by a nucleic acid comprising SEQ ID NO: 18.

In one example, the disclosure provides an anti-SARS-COV-2 antibody comprising a V_{H} comprising a sequence expressed from or encoded by a nucleic acid comprising SEQ ID NO: 11 and a V_{L} comprising a sequence expressed from or encoded by a nucleic acid comprising SEQ ID NO: 12.

In one example, the disclosure provides an anti-SARS-COV-2 antibody comprising a V_{H} comprising a sequence expressed from or encoded by a nucleic acid comprising SEQ ID NO: 13 and a V_{L} comprising a sequence expressed from or encoded by a nucleic acid comprising SEQ ID NO: 14.

In one example, the disclosure provides an anti-SARS-COV-2 antibody comprising a V_{H} comprising a sequence expressed from or encoded by a nucleic acid comprising SEQ ID NO: 15 and a V_{L} comprising a sequence expressed from or encoded by a nucleic acid comprising SEQ ID NO: 16.

In one example, the disclosure provides an anti-SARS-COV-2 antibody comprising a V_{H} comprising a sequence expressed from or encoded by a nucleic acid comprising SEQ ID NO: 17 and a V_{L} comprising a sequence expressed from or encoded by a nucleic acid comprising SEQ ID NO: 18.

In one example, the disclosure provides an anti-SARS-COV-2 antibody comprising a V_{H} comprising a sequence expressed from or encoded by a nucleic acid comprising SEQ ID NO: 19 and a V_{L} comprising a sequence expressed from or encoded by a nucleic acid comprising SEQ ID NO: 20.

The present disclosure further provides a polynucleotide encoding the protein or anti-SARS-COV-2 antibody described herein.

In one example, the polynucleotide comprises a nucleic acid sequence set forth in SEQ ID NO: 11 to SEQ ID NO: 20.

In one example, the polynucleotide comprises:
(i) a V_{H} comprising a nucleic acid sequence set forth in SEQ ID NO: 11 and a V_{L} comprising a nucleic acid sequence set forth in SEQ ID NO: 12; or
(ii) a V_{H} comprising a nucleic acid sequence set forth in SEQ ID NO: 13 and a V_{L} comprising a nucleic acid sequence set forth in SEQ ID NO: 14; or
(iii) a V_{H} comprising a nucleic acid sequence set forth in SEQ ID NO: 15 and a V_{L} comprising a nucleic acid sequence set forth in SEQ ID NO: 16; or
(iv) a V_{H} comprising a nucleic acid sequence set forth in SEQ ID NO: 17 and a V_{L} comprising a nucleic acid sequence set forth in SEQ ID NO: 18; or
(v) a V_{H} comprising a nucleic acid sequence set forth in SEQ ID NO: 19 and a V_{L} comprising a nucleic acid sequence set forth in SEQ ID NO: 20.

In one example, the polynucleotide comprises a V_{H} comprising a nucleic acid sequence set forth in SEQ ID NO: 11 and a V_{L} comprising a nucleic acid sequence set forth in SEQ ID NO: 12.

In one example, the polynucleotide comprises a V_{H} comprising a nucleic acid sequence set forth in SEQ ID NO: 13 and a V_{L} comprising a nucleic acid sequence set forth in SEQ ID NO: 14.

In one example, the polynucleotide comprises a V_{H} comprising a nucleic acid sequence set forth in SEQ ID NO: 15 and a V_{L} comprising a nucleic acid sequence set forth in SEQ ID NO: 16.

In one example, the polynucleotide comprises a V_{H} comprising a nucleic acid sequence set forth in SEQ ID NO: 17 and a V_{L} comprising a nucleic acid sequence set forth in SEQ ID NO: 18.

In one example, the polynucleotide comprises a V_{H} comprising a nucleic acid sequence set forth in SEQ ID NO: 19 and a V_{L} comprising a nucleic acid sequence set forth in SEQ ID NO: 20.

In one example, the polynucleotide of the disclosure is operably linked to a heterologous promoter.

The present disclosure additionally provides an expression construct comprising the nucleic acid of the disclosure operably linked to a promoter. Such an expression construct can be in a vector, e.g., a plasmid.

In examples of the disclosure directed to single polypeptide that form a protein comprising an antibody variable region, the expression construct may comprise a promoter linked to a nucleic acid encoding that polypeptide chain.

In examples directed to multiple polypeptides that form a protein comprising an antibody variable region, an expression construct of the disclosure comprises a nucleic acid encoding one of the polypeptides (e.g., comprising a V_{H}) operably linked to a promoter and a nucleic acid encoding another of the polypeptides (e.g., comprising a V_{L}) operably linked to another promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide.

For example, the first polypeptide comprises a V_{H} and the second polypeptide comprises a V_{L}, or the first polypeptide comprises a V_{L} and the second polypeptide comprises a V_{H}.

The present disclosure also contemplates separate expression constructs one of which encodes a first polypeptide (e.g., comprising a V_{H}) and another of which encodes a second polypeptide (e.g., comprising a V_{L}). For example, the present disclosure also provides a composition comprising:
(i) a first expression construct comprising a nucleic acid encoding a polypeptide (e.g., comprising a V_{H} operably linked to a promoter); and
(ii) a second expression construct comprising a nucleic acid encoding a polypeptide (e.g., comprising a V_{L} operably linked to a promoter),
   wherein the first and second polypeptides associate to form a protein comprising an antibody variable region.

The present disclosure additionally provides an isolated cell expressing the protein comprising an antibody variable region or a recombinant cell genetically-modified to express a protein or antibody of the disclosure. For example, the disclosure provides use of an isolated cell for preparing the protein or antibody of the disclosure. In another example, the cell comprises the nucleic acid of the disclosure or the expression construct of the disclosure or:
(i) a first expression construct comprising a nucleic acid encoding a polypeptide (e.g., comprising a V_{H}) operably linked to a promoter; and
(ii) a second expression construct comprising a nucleic acid encoding a polypeptide (e.g., comprising a V_{L}) operably linked to a promoter,
   wherein the first and second polypeptides associate to form a protein or antibody.

The present disclosure further provides use of the protein or antibody to detect an antigen having a conformation sufficient to induce an immune response against SARS-CoV-2.

The present disclosure also provides a method of detecting an antigen having a conformation sufficient to induce an immune response against SARS-COV-2, the method comprising contacting a protein or antibody described herein with the antigen and detecting binding of the protein or antibody to the antigen, wherein binding of the protein or antibody to the antigen indicates the antigen having a conformation sufficient to induce an immune response against SARS-COV-2.

The present disclosure further provides a kit or panel of proteins or antibodies for detecting an antigen having a conformation sufficient to induce an immune response against SARS-COV-2, the kit or panel comprising one or more proteins or antibodies described herein.

In one example, the antigen is a protein, a peptide, an attenuated virus or a virus-like particle. For example, the antigen is a protein, such as an antibody or antigen binding fragment. For example, the antigen is a peptide. For example, the antigen is an attenuated virus. For example, the antigen is a virus like particle.

The present disclosure also provides a pharmaceutical composition comprising the protein and a pharmaceutically acceptable carrier.

In one example, the carrier is pharmaceutically acceptable.

The present disclosure additionally provides the protein or the antibody or the nucleic acid or the expression construct or the cell or the composition of the present disclosure for use as a medicament.

The present disclosure additionally provides the protein, the antibody or the composition of the present disclosure for use in treating, preventing and/or delaying progression of a respiratory viral infection in a subject. In one example, the protein or the antibody or the nucleic acid or the expression construct or the cell or the composition of the present disclosure is for use in treating a respiratory viral infection in a subject. In another example, the protein or the antibody or the nucleic acid or the expression construct or the cell or the composition of the present disclosure is for use in preventing a respiratory viral infection in a subject. In a further example, the protein or the antibody or the nucleic acid or the expression construct or the cell or the composition of the present disclosure is for use in delaying progression of a respiratory viral infection in a subject.

The present disclosure also provides a method of treating, preventing and/or delaying progression of a respiratory viral infection in a subject in need thereof, the method comprising administering to the subject protein or the antibody or the nucleic acid or the expression construct or the cell or the composition of the present disclosure to the subject. For example, the disclosure provides a method of treating a respiratory viral infection in a subject. In another example, the disclosure provides a method of preventing a respiratory viral infection in a subject. In a further example, the disclosure provides a method of delaying progression of a respiratory viral infection in a subject.

The present disclosure additionally provides use of the protein or the antibody or the composition of the present disclosure in the manufacture of a medicament for treating, preventing and/or delaying progression of a respiratory viral infection in a subject in need thereof. For example, the disclosure provides for use of protein or the antibody or the composition of the present disclosure in the manufacture of a medicament for treating a respiratory viral infection in a subject. In another example, the disclosure provides for use of the protein or the antibody or the composition of the present disclosure in the manufacture of a medicament for preventing a respiratory viral infection in a subject. In a further example, the disclosure provides for use of the protein or the antibody or the composition of the present disclosure in the manufacture of a medicament for delaying progression of a respiratory viral infection in a subject in need thereof.

In one example, the subject is suffering from a respiratory viral infection (i.e., the subject is in need of treatment).

In one example, the respiratory viral infection is selected from the group consisting of a SARS-COV-2 infection, coronavirus disease 2019 (COVID-19) and acute respiratory disease syndrome (ARDS) and combinations thereof.

In one example, the respiratory viral infection is a SARS-COV-2 infection.

In one example, the respiratory viral infection is COVID-19.

In one example, the respiratory viral infection is ARDS.

In one example, the respiratory viral infection is a SARS-COV-2 infection and COVID-19.

In one example, the respiratory viral infection is COVID-19 and ARDS.

In one example, the present disclosure provides the pharmaceutical composition of the disclosure for use in treating, preventing and/or delaying progression of a SARS-CoV-2 infection. For example, the disclosure provides the pharmaceutical composition of the disclosure for use in treating a SARS-COV-2 infection. In another example, the disclosure provides the pharmaceutical composition of the disclosure for use in preventing a SARS-COV-2 infection. In another example, the disclosure provides the pharmaceutical composition of the disclosure for use in delaying progression of a SARS-CoV-2 infection.

In one example, the present disclosure provides a pharmaceutical composition of the disclosure for use in treating, preventing and/or delaying progression of COVID-19. For example, the disclosure provides the pharmaceutical composition of the disclosure for use in treating COVID-19. In another example, the disclosure provides the pharmaceutical composition of the disclosure for use in preventing COVID-19. In another example, the disclosure provides the pharmaceutical composition of the disclosure for use in delaying progression of COVID-19.

In one example, the present disclosure provides the pharmaceutical composition of the disclosure for use in treating, preventing and/or delaying progression of ARDS. For example, the disclosure provides the pharmaceutical composition of the disclosure for use in treating of ARDS. In another example, the disclosure provides the pharmaceutical composition of the disclosure for use in the prevention of ARDS. In another example, the disclosure provides the pharmaceutical composition of the disclosure for use in delaying the progression of ARDS.

In one example of any method described herein, the protein, the antibody, or the pharmaceutical composition of the present disclosure is administered before or after the development of a SARS-COV-2 infection, COVID-19 and/or ARDS in a subject. In one example of any method described herein, the protein, the antibody, or the pharmaceutical composition of the present disclosure is administered before the development of a SARS-CoV-2 infection, COVID-19 and/or ARDS in a subject. In one example of any method described herein, the protein, the antibody, or the pharmaceutical composition of the present disclosure is administered after the development of a SARS-COV-2 infection, COVID-19 and/or ARDS in a subject.

In one example of any method described herein, the protein, the antibody, or the pharmaceutical composition of the present disclosure is administered after the detection of a respiratory viral infection. For example, the protein, the antibody, or the pharmaceutical composition of the present disclosure is administered after the detection of a SARS-COV-2 infection, COVID-19 and/or ARDS in a subject. In another example of any method described herein, the protein, the antibody, or the pharmaceutical composition of the present disclosure is administered after the detection of a SARS-COV-2 infection. In one example, the protein, the antibody, or the pharmaceutical composition of the present disclosure is administered after the detection of a SARS-COV-2 infection but prior to the development of COVID-19. In one example, the protein, the antibody, or the pharmaceutical composition of the present disclosure is administered after the detection of ARDS.

In one example, the subject is at risk of developing a SARS-COV-2 infection, COVID-19 and/or ARDS. For example, the subject is at risk of developing a SARS-CoV-2 infection. In another example, the subject is at risk of developing COVID-19. In a further example, the subject is at risk of developing ARDS.

In one example, the composition of the present disclosure is administered in an amount sufficient to reduce the severity of or prevent onset of one or more symptoms of a SARS-COV-2 infection, COVID-19 and/or ARDS. Symptoms of a SARS-COV-2 infection, COVID-19 and/or ARDS will be apparent to the skilled person and/or are described herein.

The present disclosure provides a method of inducing an immune response in a subject, comprising administering the protein, the antibody or the pharmaceutical composition of the present disclosure to a subject in need thereof.

The present disclosure also provides use of the protein, the antibody or the pharmaceutical composition of the present disclosure in the manufacture of a medicament for inducing an immune response in a subject in need thereof.

In one example, the method or the use comprises administering to a subject in need thereof the protein, the antibody and/or the pharmaceutical compositions of the present disclosure.

In one example, the method or the use comprises administering to a subject in need thereof a plurality of proteins, antibodies, and/or pharmaceutical compositions of the present disclosure.

In one example, the method or use comprises administering to a subject in need thereof a plurality of proteins, antibodies and/or pharmaceutical compositions of the disclosure, wherein the plurality of proteins, antibodies and/or compositions are the same.

In one example, the method or use comprises administering to a subject in need thereof a plurality of proteins, antibodies and/or pharmaceutical compositions of the disclosure, wherein the plurality of proteins, antibodies and/or compositions are different. For example, the method or use comprises administering an initial and subsequent protein, antibody and/or pharmaceutical composition to the subject.

In one example, the plurality of proteins, antibodies and/or compositions are administered at the same time or sequentially. For example, a plurality of proteins and/or antibodies are formulated in the same composition. In another example, a plurality of proteins and/or antibodies are formulated in separate compositions which are administered at the same time or sequentially.

In one example, the plurality of proteins, antibodies and/or compositions are administered at different time points. In one example, a first and a second and/or subsequent dose is administered at defined intervals, for example about 24-28 weeks apart or about 48-56 weeks apart. For example, each dose is administered at intervals each of about 24-26 weeks or about 38-42 weeks, or about 50-54 weeks.

In one example, the plurality of proteins, antibodies and/or compositions are formulated separately and administered to a subject at separate time intervals. In one example, the plurality of proteins, antibodies and/or compositions are administered at separate time points but before the development of a respiratory viral infection. In another example, a first protein, antibody or composition is administered to a subject before the development of a respiratory viral infection and a second protein, antibody or composition is administered to the subject after the development of a respiratory viral infection. In a further example, the plurality of proteins, antibodies and/or compositions are administered at separate time points but after the development of a respiratory viral infection. For example, a first protein, antibody or composition is administered to a subject after the development of a SARS-COV-2 viral infection and a second protein, antibody or composition is administered to the subject after the development of a SARS-CoV-2 viral infection but before the development of COVID-19 and/or ARDS.

In one example, the protein, the antibody or the pharmaceutical composition of the present disclosure induces a cell-mediated immune response. Methods of determining whether a protein induces a cell-mediated immune response will be apparent to the skilled person and/or are described herein. For example, the cell-mediated immune response includes assessing activation of antigen-specific cytotoxic T cells. In one example, the T cells are CD4 T cells and/or CD8 T cells.

In one example, administration of the protein, the antibody or the pharmaceutical composition of the present disclosure induces a CD4 T cell mediated immune response.

In one example, administration of the protein, the antibody or the pharmaceutical composition of the present disclosure induces a CD8 T cell mediated immune response.

In one example, administration of the protein, the antibody or the pharmaceutical composition of the present disclosure induces a CD4 and CD8 T cell mediated immune response.

In one example of any method described herein, the subject is a mammal, for example a primate such as a human.

The present disclosure also provides a kit comprising at least one protein or antibody of the disclosure, optionally in a delivery system and/or a pharmaceutically acceptable carrier or diluent, packaged with instructions for use in treating or preventing a respiratory viral infection (e.g., a SARS-COV-2 infection, COVID-19 and/or ARDS) in a subject

The present disclosure also provides a kit comprising at least one protein or antibody of the disclosure, optionally in a delivery system and/or a pharmaceutically acceptable carrier or diluent, packaged with instructions to administer the protein, the antibody or the pharmaceutical composition to a subject who is suffering from or at risk of suffering from a respiratory viral infection (e.g., a SARS-COV-2 infection, COVID-19 and/or ARDS).

The present disclosure also provides a kit comprising a plurality of proteins or antibodies of the disclosure, optionally in a delivery system and/or a pharmaceutically acceptable carrier or diluent, packaged with instructions to administer the protein, the antibody or the pharmaceutical composition to a subject who is suffering from or at risk of suffering from a respiratory viral infection (e.g., a SARS-COV-2 infection, COVID-19 and/or ARDS).

In one example, the protein, the antibody, or the pharmaceutical composition of the disclosure is supplied in a vial. In another example, the protein, the antibody, or the pharmaceutical composition of the disclosure is supplied in a syringe.

### Key to Sequence Listing

SEQ ID NO: 1 amino acid sequence of a V_{H} of antibody 6G6
SEQ ID NO: 2 amino acid sequence of a V_{L} of antibody 6G6
SEQ ID NO: 3 amino acid sequence of a V_{H} of antibody 19G2
SEQ ID NO: 4 amino acid sequence of a V_{L} of antibody 19G2
SEQ ID NO: 5 amino acid sequence of a V_{H} of antibody 30B8
SEQ ID NO: 6 amino acid sequence of a V_{L} of antibody 30B8
SEQ ID NO: 7 amino acid sequence of a V_{H} of antibody 39E7
SEQ ID NO: 8 amino acid sequence of a V_{L} of antibody 39E7
SEQ ID NO: 9 amino acid sequence of a V_{H} of antibody 33E10
SEQ ID NO: 10 amino acid sequence of a V_{L} of antibody 33E10
SEQ ID NO: 11 nucleotide sequence of a V_{H} of antibody 6G6
SEQ ID NO: 12 nucleotide sequence of a V_{L} of antibody 6G6
SEQ ID NO: 13 nucleotide sequence of a V_{H} of antibody 19G2
SEQ ID NO: 14 nucleotide sequence of a V_{L} of antibody 19G2
SEQ ID NO: 15 nucleotide sequence of a V_{H} of antibody 30B8
SEQ ID NO: 16 nucleotide sequence of a V_{L} of antibody 30B8
SEQ ID NO: 17 nucleotide sequence of a V_{H} of antibody 39E7
SEQ ID NO: 18 nucleotide sequence of a V_{L} of antibody 39E7
SEQ ID NO: 19 nucleotide sequence of a V_{H} of antibody 33E10
SEQ ID NO: 20 nucleotide sequence of a V_{L} of antibody 33E10
SEQ ID NO: 21 amino acid sequence of a V_{H} CDR1 of antibody 6G6
SEQ ID NO: 22 amino acid sequence of a V_{H} CDR2 of antibody 6G6
SEQ ID NO: 23 amino acid sequence of a V_{H} CDR3 of antibody 6G6
SEQ ID NO: 24 amino acid sequence of a V_{L} CDR1 of antibody 6G6
SEQ ID NO: 25 amino acid sequence of a V_{L} CDR2 of antibody 6G6
SEQ ID NO: 26 amino acid sequence of a V_{L} CDR3 of antibody 6G6
SEQ ID NO: 27 amino acid sequence of a V_{H} CDR1 of antibody 19G2
SEQ ID NO: 28 amino acid sequence of a V_{H} CDR2 of antibody 19G2
SEQ ID NO: 29 amino acid sequence of a V_{H} CDR1 of antibodies 30B8 and 39E7
SEQ ID NO: 30 amino acid sequence of a V_{H} CDR2 of antibodies 30B8 and 39E7
SEQ ID NO: 31 amino acid sequence of a V_{H} CDR3 of antibody 30B8
SEQ ID NO: 32 amino acid sequence of a V_{L} CDR1 of antibody 30B8
SEQ ID NO: 33 amino acid sequence of a V_{L} CDR2 of antibody 30B8
SEQ ID NO: 34 amino acid sequence of a V_{L} CDR3 of antibody 30B8
SEQ ID NO: 35 amino acid sequence of a V_{H} CDR3 of antibodies 39E7 and 19G2
SEQ ID NO: 36 amino acid sequence of a V_{L} CDR1 of antibodies 39E7 and 19G2
SEQ ID NO: 37 amino acid sequence of a V_{L} CDR2 of antibodies 39E7 and 19G2
SEQ ID NO: 38 amino acid sequence of a V_{L} CDR3 of antibodies 39E7 and 19G2
SEQ ID NO: 39 amino acid sequence of a V_{H} CDR1 of antibodies 33E10
SEQ ID NO: 40 amino acid sequence of a V_{H} CDR2 of antibodies 33E10
SEQ ID NO: 41 amino acid sequence of a V_{H} CDR3 of antibodies 33E10
SEQ ID NO: 42 amino acid sequence of a V_{L} CDR1 of antibodies 33E10
SEQ ID NO: 43 amino acid sequence of a V_{L} CDR2 of antibodies 33E10
SEQ ID NO: 44 amino acid sequence of a V_{L} CDR3 of antibodies 33E10
SEQ ID NO: 45 amino acid consensus sequence of a V_{H} of antibodies 30B8, 39E7 and 19G2
SEQ ID NO: 46 amino acid consensus sequence of a V_{L} of antibodies 39E7 and 19G2
SEQ ID NO: 47 amino acid consensus sequence of a V_{H} CDR1 of antibodies 30B8, 39E7 and 19G2
SEQ ID NO: 48 amino acid consensus sequence of a V_{H} CDR2 of antibodies 30B8, 39E7 and 19G2
SEQ ID NO: 49 amino acid consensus sequence of a V_{H} CDR3 of antibodies 30B8, 39E7 and 19G2
SEQ ID NO: 50 amino acid sequence to SARS-COV-2 S protein full length (wt)
SEQ ID NO: 51 amino acid sequence to peptide 40

### BRIEF DESCRIPTION OF THE DRAWINGS

FIG. 1 is a series of graphical representations showing the concentration of monoclonal antibody clones (A) 6G6 and (B) 30B8 required to neutralize wild type and mutant SARS-COV-2 S protein. Data expressed as 50% inhibitory concentration (IC₅₀) as determined by pseudovirus assay.

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

Any discussion of a protein or antibody herein will be understood to include any variants of the protein or antibody produced during manufacturing and/or storage. For example, during manufacturing or storage an antibody can be deamidated (e.g., at an asparagine or a glutamine residue) and/or have altered glycosylation and/or have a glutamine residue converted to pyroglutamate and/or have a N-terminal or C-terminal residue removed or “clipped” and/or have part or all of a signal sequence incompletely processed and, as a consequence, remain at the terminus of the antibody. It is understood that a composition comprising a particular amino acid sequence may be a heterogeneous mixture of the stated or encoded sequence and/or variants of that stated or encoded sequence.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

As used herein the term “derived from” shall be taken to indicate that a specified integer may be obtained from a particular source albeit not necessarily directly from that source.

### Selected Definitions

As used herein, the term “severe acute respiratory syndrome coronavirus 2 (SARS-COV-2)” also known as “2019 novel coronavirus (2019-nCOV)” and “human coronavirus 2019 (HCoV-19 or hCoV-19)” will be understood to refer to a strain of coronavirus that causes coronavirus disease 2019 (COVID-19).

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulfide bond. Examples of non-covalent bonds include hydrogen bonds, ionic bonds, Van der Waals forces, and hydrophobic interactions.

As used herein, the term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

As used herein, the term “recombinant” shall be understood to mean the product of artificial genetic recombination.

As used herein, the term “antibody” will be understood to mean a protein that comprises a variable region made up of a plurality of polypeptide chains, e.g., a polypeptide comprising a light chain variable region (V_{L}) and a polypeptide comprising a heavy chain variable region (V_{H}). An antibody also generally comprises constant domains, some of which can be arranged into a constant region, which includes a constant fragment or fragment crystallizable (Fc), in the case of a heavy chain. A V_{H} and a V_{L} interact to form a Fv comprising an antigen binding region that is capable of specifically binding to one or a few closely related antigens. Generally, a light chain from mammals is either a κ light chain or a λ light chain and a heavy chain from mammals is α, δ, ε, γ, or μ. Antibodies can be of any type (e.g., IgG, IgE, IgM, IgD, IgA, and IgY), class (e.g., IgG₁, IgG₂, IgG₃, IgG₄, IgA₁ and IgA₂) or subclass. The term “antibody” also encompasses humanized antibodies, primatized antibodies, human antibodies, synhumanized antibodies and chimeric antibodies.

As used herein, the term terms “full-length antibody,” “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and includes amino acid sequences of complementarity determining regions (CDRs); i.e., CDR1, CDR2, and CDR3, and framework regions (FRs). Exemplary variable regions comprise three or four FRs (e.g., FR1, FR2, FR3 and optionally FR4) together with three CDRs. In the case of a protein derived from an IgNAR, the protein may lack a CDR2. V_{H} refers to the variable region of the heavy chain. V_{L} refers to the variable region of the light chain.

As used herein, the term “complementarity determining regions” (syn. CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. The amino acid positions assigned to CDRs and FRs can be defined according to Kabat Sequences of Proteins of Immunological Interest, National Institutes of Health, Bethesda, Md., 1987 and 1991 or other numbering systems in the performance of this disclosure, e.g., the canonical numbering system of Chothia and Lesk J. Mol Biol. 196:901-917, 1987; Chothia et al. Nature 342, 877-883, 1989; and/or Al-Lazikani et al., J Mol Biol 273:927-948, 1997; the IMGT numbering system of Lefranc et al., Devel. And Compar. Immunol., 27:55-77, 2003; or the AHO numbering system of Honnegher and Plükthun J. Mol. Biol., 309:657-670, 2001.

As used herein, the term “Framework regions” (FRs) are those variable domain residues other than the CDR residues.

As used herein, the term “Fv” shall be taken to mean any protein, whether comprised of multiple polypeptides or a single polypeptide, in which a V_{L} and a V_{H} associate and form a complex having an antigen binding site, i.e., capable of specifically binding to an antigen. The V_{H} and the V_{L} which form the antigen binding site can be in a single polypeptide chain or in different polypeptide chains. Furthermore, an Fv of the disclosure (as well as any protein of the disclosure) may have multiple antigen binding sites which may or may not bind the same antigen. This term shall be understood to encompass fragments directly derived from an antibody as well as proteins corresponding to such a fragment produced using recombinant means. In some examples, the V_{H} is not linked to a heavy chain constant domain (C_{H}) 1 and/or the V_{L} is not linked to a light chain constant domain (C_{L}). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., C_{H}2 or C_{H}3 domain, e.g., a minibody. A “Fab fragment” consists of a monovalent antigen-binding fragment of an antibody, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a V_{H} and a single constant domain. Two Fab′ fragments are obtained per antibody treated in this manner. A Fab′ fragment can also be produced by recombinant means. A “F(ab′)₂ fragment” of an antibody consists of a dimer of two Fab′ fragments held together by two disulfide bonds, and is obtained by treating a whole antibody molecule with the enzyme pepsin, without subsequent reduction. A “Fab₂” fragment is a recombinant fragment comprising two Fab fragments linked using, for example a leucine zipper or a C_{H}3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

As used herein, the term “binds” in reference to the interaction of a protein or an antigen binding site thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabeled “A”), in a reaction containing labeled “A” and the protein, will reduce the amount of labeled “A” bound to the antibody.

As used herein, the term “specifically binds” or “binds specifically” shall be taken to mean that a protein of the disclosure reacts or associates more frequently, more rapidly, with greater duration and/or with greater affinity with a particular antigen or cell expressing same than it does with alternative antigens or cells. For example, a protein binds to SARS-COV-2 S protein with materially greater affinity (e.g., 1.5 fold or 2 fold or 5 fold or 10 fold or 20 fold or 40 fold or 60 fold or 80 fold to 100 fold or 150 fold or 200 fold) than it does to antigens commonly recognized by polyreactive natural antibodies (i.e., by naturally occurring antibodies known to bind a variety of antigens naturally found in humans). Generally, but not necessarily, reference to binding means specific binding, and each term shall be understood to provide explicit support for the other term.

As used herein, the term “neutralize” shall be taken to mean that a protein is capable of blocking, reducing or preventing binding of SARS-COV-2S protein RBD to angiotensin-converting enzyme 2 (ACE2). It will be apparent from the foregoing that the protein need not completely neutralize binding of S protein RBD to ACE2, rather it need only neutralize binding by a statistically significant amount, for example, by at least about 30% or 40% or 50% or 60% or 70% or 80% or 90% or 95% as determined by a neutralisation assay, including for example sVNT PsV and Vero microneutralisation assay

As used herein, the term “antigen” refers to a molecule or structure containing one or more epitopes that induce, elicit, augment or boost a cellular and/or humoral immune response.

The term “competitively inhibits” shall be understood to mean that a protein of the disclosure (or an antigen binding site thereof) reduces or prevents binding of a recited antibody or protein to SARS-COV-2 S protein RBD. This may be due to the protein (or antigen binding site) and antibody binding to the same or an overlapping epitope. It will be apparent from the foregoing that the protein need not completely inhibit binding of the antibody, rather it need only reduce binding by a statistically significant amount, for example, by at least about 10% or 20% or 30% or 40% or 50% or 60% or 70% or 80% or 90% or 95%. Preferably, the protein reduces binding of the antibody by at least about 30%, more preferably by at least about 50%, more preferably, by at least about 70%, still more preferably by at least about 75%, even more preferably, by at least about 80% or 85% and even more preferably, by at least about 90%. Methods for determining competitive inhibition of binding are known in the art and/or described herein. For example, the antibody is exposed to SARS-COV-2 S protein RBD either in the presence or absence of the protein. If less antibody binds in the presence of the protein than in the absence of the protein, the protein is considered to competitively inhibit binding of the antibody. In one example, the competitive inhibition is not due to steric hindrance.

“Overlapping” in the context of two epitopes shall be taken to mean that two epitopes share a sufficient number of amino acid residues to permit a protein (or antigen binding site thereof) that binds to one epitope to competitively inhibit the binding of a protein (or antigen binding site) that binds to the other epitope. For example, the “overlapping” epitopes share at least 1 or 2 or 3 or 4 or 5 or 6 or 7 or 8 or 9 or 20 amino acids.

As used herein, the term “half-maximal inhibitory concentration (IC₅₀)” refers to a concentration of a compound (e.g. a protein described herein) that is required to inhibit a biological process by half. For example, at least about 5 μg/ml of a protein described herein neutralises binding of S protein RBD to ACE transfected VeroE6 cells by half.

As used herein, the term “epitope” (syn. “antigenic determinant”) shall be understood to mean a region of S protein RBD to which a protein comprising an antibody variable region binds. This term is not necessarily limited to the specific residues or structure to which the protein makes contact. For example, this term includes the region spanning amino acids contacted by the protein and/or 5-10 or 2-5 or 1-3 amino acids outside of this region. In some examples, the epitope comprises a series of discontinuous amino acids that are positioned close to one another when S protein RBD is folded, i.e., a “conformational epitope”. The skilled artisan will also be aware that the term “epitope” is not limited to peptides or polypeptides. For example, the term “epitope” includes chemically active surface groupings of molecules such as sugar side chains, phosphoryl side chains, or sulfonyl side chains, and, in certain examples, may have specific three dimensional structural characteristics, and/or specific charge characteristics.

As used herein, the term “cross-reacts” or “cross-reactivity” will be understood to mean that a protein raised against a specific antigen has a competing affinity to another antigen (e.g., a mutant form of the antigen).

As used herein, the term “nucleotide sequence” or “nucleic acid sequence” will be understood to mean a series of contiguous nucleotides (or bases) covalently linked to a phosphodiester backbone.

As used herein, a subject “at risk” of developing a respiratory viral infection may or may not have detectable symptoms of a respiratory viral infection, and may or may not have displayed detectable symptoms of a respiratory viral infection prior to the treatment according to the present disclosure. “At risk” denotes that a subject has one or more risk factors, which are measurable parameters that correlate with development of the respiratory viral infection, as known in the art and/or described herein.

As used herein, the term “subject” shall be taken to mean any animal include humans, for example, a mammal. Exemplary subjects include but are not limited to humans and non-human primates. For example, the subject is a human.

As used herein, the terms “treating”, “treat”, “treatment” or “treating” include administering a protein, an antibody or pharmaceutical composition described herein to thereby reduce or eliminate at least one symptom of a respiratory viral infection in a subject.

As used herein, the term “preventing”, “prevent” or “prevention” includes providing prophylaxis with respect to occurrence or recurrence of a specified respiratory viral infection in a subject. A subject may be predisposed to or at risk of developing a respiratory viral infection but has not yet been diagnosed with the respiratory viral infection.

As used herein, the phrase “delaying progression of” includes reducing or slowing down the progression of a respiratory viral infection in a subject and/or at least one symptom of the respiratory viral infection.

An “effective amount” refers to at least an amount effective, at dosages and for periods of time necessary, to achieve the desired result. For example, the desired result may be a therapeutic or prophylactic result. An effective amount can be provided in one or more administrations. In some examples of the present disclosure, the term “effective amount” is meant an amount necessary to effect in treating a respiratory viral infection as hereinbefore described. In some examples of the present disclosure, the term “effective amount” is meant an amount necessary to effect a change associated with a disease or condition as hereinbefore described. The effective amount may vary according to the disease or condition to be treated or factor to be altered and also according to the weight, age, racial background, sex, health and/or physical condition and other factors relevant to the mammal being treated. Typically, the effective amount will fall within a relatively broad range (e.g. a “dosage” range) that can be determined through routine trial and experimentation by a medical practitioner. Accordingly, this term is not to be construed to limit the disclosure to a specific quantity, e.g., weight or number of binding proteins. The effective amount can be administered in a single dose or in a dose repeated once or several times over a treatment period.

A “therapeutically effective amount” is at least the minimum concentration required to effect a measurable improvement of a particular disease or condition. A therapeutically effective amount herein may vary according to factors such as the disease state, age, sex, and weight of the patient, and the ability of the protein or antibody of the present disclosure to elicit a desired response in the individual. A therapeutically effective amount is also one in which any toxic or detrimental effects of the molecule are outweighed by the therapeutically beneficial effects.

As used herein, the term “prophylactically effective amount” shall be taken to mean a sufficient quantity of the molecule of the disclosure to prevent or inhibit or delay the onset of one or more detectable symptoms of a disease or disorder as described herein.

### SARS-COV-2 Spike Protein

SARS-COV-2 is a member of the Coronaviridae family of enveloped, positive-sense single-stranded RNA viruses. The SARS-COV-2 protein comprises four structural proteins: spike(S), membrane (M), nucleocapsid (N) and envelope (E).

The S protein is responsible for recognizing the target angiotensin converting enzyme 2 (ACE2) receptor and mediating fusion of the virus and the target cell membrane, which is considered as key to the infection process. The S protein is a large type I transmembrane protein that is highly glycosylated. It contains two subunits, S1 and S2. There are two important domains in S1 subunits, known as the N-Terminal Domain (NTD) and the Receptor Binding Domain (RBD), which is responsible for binding to ACE2. S2 has three domains called Heptad Repeat (HR), Central Helix (C_{H}), and Connector Domain (CD) respectively. Additionally, there is a furin cleavage site at S1/S2.

The S protein protrudes from the viral surface as a homotrimer with two different conformations, pre-fusion and post-fusion. It is the trimeric assembly of the S protein on the virion surface that gives it the distinctive “corona” or crown-like appearance. The binding of the S protein RBD to ACE2 triggers the structural change from pre- to post-fusion, resulting in dissociation of the S1 and S2 subunits and transformation of the S2 subunit into a highly stable post-fusion conformation.

The present disclosure provides a protein which binds to SARS-COV-2 S protein RBD and neutralises binding of the S protein RBD to ACE2. In one example, the protein competes with an antibody described herein to neutralises binding of SARS-COV-2 RBD to ACE2.

### Proteins Comprising an Antibody Variable Region

The present disclosure provides a protein comprising an antibody variable region that binds specifically to a S protein RBD and neutralises binding of the S protein to ACE2.

The present disclosure also provides use of the protein to detect an antigen having a conformation sufficient to induce an immune response against SARS-COV-2.

### Antibodies

The present disclosure provides an anti-SARS-COV-2 antibody, e.g., comprising the variable regions described herein. For example, the disclosure provides an anti-SARS-COV-2 neutralising antibody. Exemplary anti-SARS-COV-2 neutralising antibodies are 6G6, 19G2, 30B8, 39E7 and 33E10.

In one example, the antibody is a recombinant antibody. For example, an antibody or protein comprising a variable region thereof is produced using a standard method, e.g., as is known in the art or briefly described herein.

Monoclonal antibodies are exemplary antibodies contemplated by the present disclosure. The term “monoclonal antibody” or “mAb” or “MAb” refers to a homogeneous antibody population capable of binding to the same antigen(s) and, for example, to the same epitope within the antigen. This term is not intended to be limited with respect to the source of the antibody or the manner in which it is made.

### Deimmunized, Chimeric, Humanized, Synhumanized, Primatized and Human Proteins

The proteins of the present disclosure may be a humanized protein.

The term “humanized protein” shall be understood to refer to a protein comprising a human-like variable region, which includes CDRs from an antibody from a non-human species (e.g., mouse or rat or non-human primate) grafted onto or inserted into FRs from a human antibody (this type of antibody is also referred to a “CDR-grafted antibody”). Humanized proteins also include proteins in which one or more residues of the human protein are modified by one or more amino acid substitutions and/or one or more FR residues of the human protein are replaced by corresponding non-human residues. Humanized proteins may also comprise residues which are found in neither the human antibody or in the non-human antibody. Any additional regions of the protein (e.g., Fc region) are generally human. Humanization can be performed using a method known in the art, e.g., U.S. Pat. Nos. 5,225,539, 6,054,297, 7,566,771 or U.S. Pat. No. 5,585,089. The term “humanized protein” also encompasses a super-humanized protein, e.g., as described in U.S. Pat. No. 7,732,578.

The proteins of the present disclosure may be human proteins. The term “human protein” as used herein refers to proteins having variable and, optionally, constant antibody regions found in humans, e.g. in the human germline or somatic cells or from libraries produced using such regions. The “human” antibodies can include amino acid residues not encoded by human sequences, e.g. mutations introduced by random or site directed mutations in vitro (in particular mutations which involve conservative substitutions or mutations in a small number of residues of the protein, e.g. in 1, 2, 3, 4 or 5 of the residues of the protein). These “human antibodies” do not necessarily need to be generated as a result of an immune response of a human, rather, they can be generated using recombinant means (e.g., screening a phage display library) and/or by a transgenic animal (e.g., a mouse) comprising nucleic acid encoding human antibody constant and/or variable regions and/or using guided selection (e.g., as described in or U.S. Pat. No. 5,565,332). This term also encompasses affinity matured forms of such antibodies. For the purposes of the present disclosure, a human protein will also be considered to include a protein comprising FRs from a human antibody or FRs comprising sequences from a consensus sequence of human FRs and in which one or more of the CDRs are random or semi-random, e.g., as described in U.S. Pat. No. 6,300,064 and/or U.S. Pat. No. 6,248,516.

The proteins of the present disclosure may be synhumanized proteins. The term “synhumanized protein” refers to a protein prepared by a method described in WO2007/019620. A synhumanized protein includes a variable region of an antibody, wherein the variable region comprises FRs from a New World primate antibody variable region and CDRs from a non-New World primate antibody variable region. For example, a synhumanized protein includes a variable region of an antibody, wherein the variable region comprises FRs from a New World primate antibody variable region and CDRs from a mouse or rat antibody.

The proteins of the present disclosure may be primatized proteins. A “primatized protein” comprises variable region(s) from an antibody generated following immunization of a non-human primate (e.g., a cynomolgus macaque). Optionally, the variable regions of the non-human primate antibody are linked to human constant regions to produce a primatized antibody. Exemplary methods for producing primatized antibodies are described in U.S. Pat. No. 6,113,898.

In one example a protein of the disclosure is a chimeric protein. The term “chimeric proteins” refers to proteins in which an antigen binding domain is from a particular species (e.g., murine, such as mouse or rat) or belonging to a particular antibody class or subclass, while the remainder of the protein is from a protein derived from another species (such as, for example, human or non-human primate) or belonging to another antibody class or subclass. In one example, a chimeric protein is a chimeric antibody comprising a V_{H} and/or a V_{L} from a non-human antibody (e.g., a murine antibody) and the remaining regions of the antibody are from a human antibody. The production of such chimeric proteins is known in the art, and may be achieved by standard means (as described, e.g., in U.S. Pat. Nos. 6,331,415; 5,807,715; 4,816,567 and 4,816,397).

The present disclosure also contemplates a deimmunized protein, e.g., as described in WO2000/34317 and WO2004/108158. De-immunized antibodies and proteins have one or more epitopes, e.g., B cell epitopes or T cell epitopes removed (i.e., mutated) to thereby reduce the likelihood that a subject will raise an immune response against the antibody or protein.

### Antibody Fragments

Single Chain Fv (scFv) Fragments and Dimeric-scFv (Di-scFv)

The skilled artisan will be aware that scFvs comprise V_{H} and V_{L} regions in a single polypeptide chain. The polypeptide chain further comprises a polypeptide linker between the V_{H} and V_{L} which enables the scFv to form the desired structure for antigen binding (i.e., for the V_{H} and V_{L} of the single polypeptide chain to associate with one another to form a Fv). For example, the linker comprises in excess of 12 amino acid residues with (Gly₄Ser)₃ being one of the more favoured linkers for a scFv.

The present disclosure also contemplates a disulfide stabilized Fv (or diFv or dsFv), in which a single cysteine residue is introduced into a FR of V_{H} and a FR of V_{L} and the cysteine residues linked by a disulfide bond to yield a stable Fv (see, for example, Brinkmann et al., 1993).

Alternatively, or in addition, the present disclosure provides a dimeric scFv, i.e., a protein comprising two scFv molecules linked by a non-covalent or covalent linkage, e.g., by a leucine zipper domain (e.g., derived from Fos or Jun) (see, for example, Kruif and Logtenberg, 1996). Alternatively, two scFvs are linked by a peptide linker of sufficient length to permit both scFvs to form and to bind to an antigen, e.g., as described in US20060263367.

For a review of scFv, see Plückthun (1994).

### Diabodies, Triabodies, Tetrabodies

Exemplary proteins comprising an antibody antigen binding domain are diabodies, triabodies, tetrabodies and higher order protein complexes such as those described in WO98/044001 and WO94/007921.

For example, a diabody is a protein comprising two associated polypeptide chains, each polypeptide chain comprising the structure V_{L}-X-V_{H} Or V_{H}-X-V_{L}, wherein V_{L} is an antibody light chain variable region, V_{H} is an antibody heavy chain variable region, X is a linker comprising insufficient residues to permit the V_{H} and V_{L} in a single polypeptide chain to associate (or form an Fv) or is absent, and wherein the V_{H} of one polypeptide chain binds to a V_{L} of the other polypeptide chain to form an antigen binding site, i.e., to form an Fv molecule capable of specifically binding to one or more antigens. The V_{L} and V_{H} can be the same in each polypeptide chain or the V_{L} and V_{H} can be different in each polypeptide chain so as to form a bispecific diabody (i.e., comprising two Fvs having different specificity).

### Minibodies

The skilled artisan will be aware that a minibody comprises the V_{H} and V_{L} domains of an antibody fused to the C_{H}2 and/or C_{H}3 domain of an antibody. Optionally, the minibody comprises a hinge region between the V_{H} and a V_{L}, sometimes this conformation is referred to as a Flex Minibody. A minibody does not comprise a C_{H}1 or a C_{L}. In one example, the V_{H} and V_{L} domains are fused to the hinge region and the C_{H}3 domain of an antibody. At least one of the variable regions of said minibody binds to the S protein RBD in the manner of the disclosure. Exemplary minibodies and methods for their production are described, for example, in WO94/09817.

### Constant Domain Fusions

The present disclosure encompasses proteins comprising a variable region and a constant region or a domain(s) thereof, e.g., Fc, C_{H}2 and/or C_{H}3 domain. The skilled artisan will be aware of the meaning of the terms constant region and constant domain based on the disclosure herein and references discussed herein.

Constant region sequences useful for producing the proteins of the present disclosure may be obtained from a number of different sources. In some examples, the constant region or portion thereof of the protein is derived from a human antibody. Moreover, the constant domain or portion thereof may be derived from any antibody class, including IgM, IgG, IgD, IgA and IgE, and any antibody isotype, including IgG₁, IgG₂, IgG₃ and IgG₄.

A variety of constant region gene sequences are available in the form of publicly accessible deposits or the sequence thereof is available from publicly available databases. Constant regions can be selected having a particular effector function (or lacking a particular effector function) or with a particular modification to reduce immunogenicity.

The present disclosure also contemplates proteins comprising mutant constant regions or domains, e.g., as described in US7217797; US7217798; or US20090041770 (having increased half-life) or US2005037000 (increased ADCC).

### Protein Production

In one example, a protein or antibody of the disclosure is produced by culturing a cell line under conditions sufficient to produce the protein, e.g., as described herein and/or as is known in the art.

### Recombinant Expression

In the case of a recombinant protein, nucleic acid encoding same is placed into one or more expression construct, e.g., expression vector(s), which is/are then transfected into host cells, such as cells that can produce a disulphide bridge or bond, such as E. coli cells, yeast cells, insect cells, or mammalian cells. Exemplary mammalian cells include simian COS cells, Chinese Hamster Ovary (CHO) cells, or myeloma cells that do not otherwise produce immunoglobulin protein. Molecular cloning techniques to achieve these ends are known in the art and described, for example in Ausubel or Sambrook. A wide variety of cloning and in vitro amplification methods are suitable for the construction of recombinant nucleic acids. Methods of producing recombinant antibodies are also known in the art. See U.S. Pat. Nos. 4,816,567; 7,923,221 and 7,022,500.

Following isolation, the nucleic acid encoding a protein of the disclosure is inserted into an expression construct or replicable vector for further cloning (amplification of the DNA) or for expression in a cell-free system or in cells. For example, the nucleic acid is operably linked to a promoter.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein of the present disclosure (e.g., derived from the information provided herein), an enhancer element, a promoter, and a transcription termination sequence. The skilled artisan will be aware of suitable sequences for expression of a protein. For example, exemplary signal sequences include prokaryotic secretion signals (e.g., pelB, alkaline phosphatase, penicillinase, Ipp, or heat-stable enterotoxin II), yeast secretion signals (e.g., invertase leader, a factor leader, or acid phosphatase leader) or mammalian secretion signals (e.g., herpes simplex gD signal).

Exemplary promoters include those active in prokaryotes (e.g., phoA promoter, β-lactamase and lactose promoter systems, alkaline phosphatase, a tryptophan (trp) promoter system, and hybrid promoters such as the tac promoter).

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CVI line transformed by SV40 (COS-7, AUSTRALIAN CELL BANK CRL 1651); human embryonic kidney line (293 or 293 cells subcloned for growth in suspension culture; baby hamster kidney cells (BHK, AUSTRALIAN CELL BANK CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the (′UPI promoter, the PHO5 promoter, the nmt promoter, the RPR1 promoter, or the TEF1 promoter.

Means for introducing the isolated nucleic acid molecule or a gene construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation, viral transduction (e.g., using a lentivirus) and microparticle bombardment such as by using DNA-coated tungsten or gold particles (Agracetus Inc., WI, USA) amongst others.

The host cells used to produce the protein of the disclosure may be cultured in a variety of media, depending on the cell type used. Commercially available media such as Ham's F10 (Sigma), Minimal Essential Medium ((MEM), (Sigma), RPM1-1640 (Sigma), and Dulbecco's Modified Eagle's Medium ((DMEM), Sigma) are suitable for culturing mammalian cells. Media for culturing other cell types discussed herein are known in the art.

### Isolation of Proteins

A protein or antibody of the present disclosure can be isolated or purified.

Methods for purifying a protein or antibody of the disclosure are known in the art and/or described herein.

When using recombinant techniques, the protein or antibody of the disclosure can be produced intracellularly, in the periplasmic space, or directly secreted into the medium. If the protein is produced intracellularly, as a first step, the particulate debris, either host cells or lysed fragments, is removed, for example, by centrifugation or ultrafiltration. Where the protein is secreted into the medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The protein prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Zola (1997).

The skilled artisan will also be aware that a protein of the disclosure can be modified to include a tag to facilitate purification or detection, e.g., a poly-histidine tag, e.g., a hexa-histidine tag, or a influenza virus hemagglutinin (HA) tag, or a Simian Virus 5 (V5) tag, or a FLAG tag, or a glutathione S-transferase (GST) tag. For example, the tag is a hexa-his tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Conjugates

The present disclosure also provides conjugates of proteins described herein according to any example. For example, a protein comprising an antibody variable region is conjugated to a detectable label, a therapeutic compound, a colloid, a toxin, a nucleic acid, a peptide, a protein, a compound that increases the half-life of the protein in a subject and mixtures thereof.

As used herein, the term “conjugate” or “conjugated” shall be understood to encompass both indirect and direct binding. For example, direct conjugation includes chemical conjugation, which can be non-covalent or covalent or genetic conjugation (also referred to as “fusion”). In one example, the conjugation is covalent, e.g., a disulphide bond.

As used herein, a “detectable label” is a molecular or atomic tag or marker that generates or can be induced to generate an optical or other signal or product that can be detected visually or by using a suitable detector. Detectable labels are well known in the art and include, for example, a radiolabel, an enzyme, a fluorescent label, a luminescent label, a bioluminescent label, a magnetic label, a prosthetic group, a contrast agent and an ultrasound agent.

In one example, a protein as described herein according to any example is conjugated or linked to another protein, including another protein of the disclosure or a protein comprising an antibody variable region, such as an antibody or a protein derived therefrom, e.g., as described herein. Other proteins are not excluded. Additional proteins will be apparent to the skilled artisan and include, for example, an immunomodulator or a half-life extending protein or a peptide or other protein that binds to serum albumin amongst others.

Exemplary serum albumin binding peptides or protein are described in US20060228364 or US20080260757.

The proteins of the present disclosure can be modified to contain additional non-proteinaceous moieties that are known in the art and readily available. For example, the moieties suitable for derivatization of the protein are physiologically acceptable polymer, e.g., a water soluble polymer. Such polymers are useful for increasing stability and/or reducing clearance (e.g., by the kidney) and/or for reducing immunogenicity of a protein of the disclosure. Non-limiting examples of water soluble polymers include, but are not limited to, polyethylene glycol (PEG), polyvinyl alcohol (PVA), or propropylene glycol (PPG).

In one example, a protein as described herein according to any example comprises one or more detectable markers to facilitate detection and/or isolation. For example, the compound comprises a fluorescent label such as, for example, fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7, fluorescein (5-carboxyfluorescein-N-hydroxysuccinimide ester), rhodamine (5,6-tetramethyl rhodamine). The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm).

Alternatively, or in addition, the protein as described herein according to any example is labeled with, for example, a fluorescent semiconductor nanocrystal (as described, for example, in U.S. Pat. No. 6,306,610).

Alternatively, or in addition, the protein is labeled with, for example, a magnetic or paramagnetic compound, such as, iron, steel, nickel, cobalt, rare earth materials, neodymium-iron-boron, ferrous-chromium-cobalt, nickel-ferrous, cobalt-platinum, or strontium ferrite.

### Assaying Proteins of the Disclosure

Proteins of the present disclosure are readily screened for physical and biological activity and/or stability using methods known in the art and/or as described below.

### Binding to a SARS-CoV-2 S Protein RBD

It will be apparent to the skilled artisan from the disclosure herein that a protein of the present disclosure binds (or specifically binds) to the SARS-COV-2 S protein RBD. Methods for assessing binding to a protein are known in the art, e.g., as described in Scopes (In: Protein purification: principles and practice, Third Edition, Springer Verlag, 1994). Such a method generally involves labelling the protein and contacting it with immobilised compound. Following washing to remove non-specific bound protein, the amount of label and, as a consequence, bound protein is detected. Of course, the protein can be immobilised and the compound that binds to RBD of SARS-COV-2 spike protein labelled. Panning-type assays can also be used. Alternatively, or additionally, surface plasmon resonance assays can be used.

The assays described above can also be used to detect the level of binding of a protein of the present disclosure to RBD of SARS-COV-2 spike protein. Methods of detecting the level of binding will be apparent to the skilled person and/or described herein. For example, the level of binding is determined using a biosensor.

### Neutralising Assays

Proteins of the disclosure may be screened in vitro for their ability to bind to a SARS-COV-2 S protein RBD and neutralises binding of the S protein RBD to ACE2. Suitable assays will be apparent to the skilled person and include, for example, a Vero microneutralisation assay, a sVNT assay, or a psuedovirus neutralisation assay (using e.g., HEK-293T cells or HeLa-ACE2 cells).

In one example, the neutralization assay is a Vero microneutralization assay. Briefly, SARS-Cov-2 wild-type virus is passaged in Vero cells (i.e., the Vero lineage isolated from kidney epithelial cells extracted from an African green monkey). Serial two-fold dilutions of a test protein are incubated with 100 TCID₅₀ (i.e., median tissue culture infectious dose) of SARS-COV-2 for 1 hour and residual virus infectivity is assessed in Vero cells; viral cytopathic effect is read, for example, on day 5. The neutralising antibody titre is calculated using the Reed/Muench method as previously described (Houser et al., 2016; Subbarao et al 2004).

In one example, the neutralization assay is a surrogate neutralization test (sVNT). Briefly, the wells of a plate are coated with hACE2 protein in carbonate-bicarbonate coating buffer (e.g., pH 9.6). HRP-conjugated SARS-COV-2 and HRP-conjugated SARS-COV-RBD pre-incubated with test proteins is added to the hACE2 at different concentrations and incubated, for example, for 1 h at room temperature. Unbound HRP conjugated antigens are removed by washing. Colorimetric signal is developed on the enzymatic reaction of HRP with chromogenic substrate, e.g., 3,3′,5,5′-tetramethylbenzidine (TMB). In one example, the absorbance reading at 450 nm and 570 nm is acquired.

In one example, the neutralisation is a psuedovirus neutralisation assay. Briefly, HIV reporter virus pseudotyped with SARS-2-Spike protein is produced by co-transfection of SARS-2-COV-2 spike plasmids together with a viral backbone plasmid (e.g., pDR-NL Δenv FLUC) into e.g., HEK-293T cells. Pseudovirus is harvested post transfection and clarified by filtration. Virus stock titres, reported as Relative Luciferase Units infectious dose (RLU), are calculated by limiting dilution infections in Hela-hACE2 cells measuring luciferase activity as a read-out for viral infection.

### Determining Competitive Binding

Assays for determining a protein that competitively inhibits binding of antibodies 6G6, 19G2, 30B8, 39E7 and/or 30E10 (or any other antibody described herein) will be apparent to the skilled artisan. For example, 6G6, 19G2, 30B8, 39E7 or 30E10 is conjugated to a detectable label, e.g., a fluorescent label or a radioactive label. The labelled antibody and the test protein are then mixed and contacted with SARS-COV-2 S protein RBD or a region thereof or a cell expressing same. The level of labelled 6G6, 19G2, 30B8, 39E7 or 30E10 is then determined and compared to the level determined when the labelled antibody is contacted with the SARS-COV-2 S protein RBD, region or cells in the absence of the protein. If the level of labelled 6G6, 19G2, 30B8, 39E7 or 30E10 is reduced in the presence of the test protein compared to the absence of the protein, the protein is considered to competitively inhibit binding of 6G6, 19G2, 30B8, 39E7 or 30E10 to RBD of SARS-COV-2 spike protein or a region thereof.

Optionally, the test protein is conjugated to different label to 6G6, 19G2, 30B8, 39E7 or 30E10. This alternate labelling permits detection of the level of binding of the test protein to SARS-COV-2 S protein RBD or a region thereof or the cell.

In another example, the protein is permitted to bind to SARS-COV-2 S protein RBD or a region thereof or a cell expressing same prior to contacting the SARS-COV-2 S protein RBD or a region thereof or a cell expressing the same with 6G6, 19G2, 30B8, 39E7 or 30E10. A reduction in the amount of bound 6G6, 19G2, 30B8, 39E7 or 30E10 in the presence of the protein compared to in the absence of the protein indicates that the protein competitively inhibits 6G6, 19G2, 30B8, 39E7 or 30E10 binding to RBD of SARS-COV-2 spike protein. A reciprocal assay can also be performed using labelled protein and first allowing 6G6, 19G2, 30B8, 39E7 or 30E10 to bind to RBD of SARS-CoV-2 spike protein. In this case, a reduced amount of labelled protein bound to SARS-CoV-2 S protein RBD in the presence of 6G6, 19G2, 30B8, 39E7 or 30E10 compared to in the absence of 6G6, 19G2, 30B8, 39E7 or 30E10 indicates that the protein competitively inhibits binding of 6G6, 19G2, 30B8, 39E7 or 30E10 to SARS-COV-2 S protein RBD.

### Epitope Mapping of Proteins

Assays for determining binding sites of a protein disclosed herein to one or more epitopes of SARS-COV-2 S protein RBD will be apparent to the skilled artisan. In an example, the protein is permitted to bind to a linear epitope of SARS-COV-2 S protein RBD. For example, a protein described herein is contacted with an epitope of SARS-COV-2 S protein and binding is determined by a specific assay (e.g. ELISA, Western Blotting, X-ray crystallography, 3D Electron Microscopy, Liquid chromatography-mass spectrometry). In one example, the assay is X-ray crystallography.

### Uses of the Protein, Antibody or Pharmaceutical Formulation

As discussed herein, the present disclosure provides a method of treating, preventing and/or delaying progression of a respiratory viral infection in a subject, comprising administering a protein, an antibody or a pharmaceutical formulation to the subject.

In one example, the protein, the antibody, or the pharmaceutical formulation is administered to the subject in an amount to reduce the severity of the respiratory viral infection and/or symptoms thereof in the subject.

In one example, the respiratory viral infection is selected from the group consisting of a SARS-COV-2 infection, COVID-19 and ARDS and combination thereof.

In one example, the respiratory viral infection is a SARS-COV-2 infection.

In one example, the respiratory viral infection is COVID-19.

In one example, the respiratory viral infection is ARDS.

In one example, the subject suffers from a respiratory viral infection (i.e., is in need thereof).

In one example, the subject suffers from a SARS-COV-2 infection, COVID-19, ARDS or a combination thereof.

In one example, the method additionally comprises identifying a subject suffering from a SARS-COV-2 infection, COVID-19, ARDS or a combination thereof. Methods of identifying such a subject will be apparent to the skilled person and/or are described herein.

In one example, the subject is at risk of developing a respiratory viral infection. For example, For example, the subject is at risk of developing a SARS-COV-2 infection, COVID-19, ARDS or a combination thereof. For example, the subject is at risk of developing a SARS-COV-2 infection. For example, the subject is at risk of developing cCOVID-19. For example, the subject is at risk of developing ARDS.

A subject is at risk if he or she has a higher risk of developing a respiratory viral infection than a control population. The control population may include one or more subjects selected at random from the general population (e.g., matched by age, gender, race and/or ethnicity) who have not suffered from or have a family history of a respiratory viral infection. A subject can be considered at risk for a complement mediated disorder if a “risk factor” associated with a respiratory viral infection is found to be associated with that subject. A risk factor can include any activity, trait, event or property associated with a given respiratory viral infection, for example, through statistical or epidemiological studies on a population of subjects. A subject can thus be classified as being at risk for a respiratory viral infection even if studies identifying the underlying risk factors did not include the subject specifically.

In one example, a method of the disclosure reduces any symptom of a respiratory viral infection (e.g. SARS-COV-2 infection, COVID-19, ARDS or a combination thereof).

As will be apparent to the skilled person a “reduction” in a symptom of a respiratory viral infection in a subject will be comparative to another subject who also suffers from a respiratory viral infection but who has not received treatment with a method described herein. This does not necessarily require a side-by-side comparison of two subjects. Rather population data can be relied upon. For example, a population of subjects suffering from a respiratory viral infection who have not received treatment with a method described herein (optionally, a population of similar subjects to the treated subject, e.g., age, weight, race) are assessed and the mean values are compared to results of a subject or population of subjects treated with a method described herein.

In one example, performing a method described herein according to any example of the disclosure results in enhancement of a clinical response and/or delayed disease progression.

By “clinical response” is meant an improvement in the symptoms of disease. The clinical response may be achieved within a certain time frame, for example, within or at about 8 weeks from the start of treatment with, or from the initial administration. Clinical response may also be sustained for a period of time, such as for >24 weeks, or ≥48 weeks.

### Coronavirus Disease 2019 (COVID-19)

The present disclosure provides, for example, methods of treating, preventing and/or delaying progression of COVID-19.

COVID-19 is an infectious disease caused by SARS-COV-2. It was first identified in December 2019 in Wuhan, Hubei, China, and has resulted in an ongoing pandemic. Common symptoms include fever, cough, fatigue, shortness of breath, and loss of smell and taste. While the majority of cases result in mild symptoms, some progress to ARDS.

The time from exposure to onset of symptoms is typically around five days, but may range from two to fourteen days. There are currently no vaccines nor specific antiviral treatments for COVID-19 and management involves the treatment of symptoms, supportive care, isolation, and experimental measures.

Thus, in some examples, the subject has a SARS-COV-2 infection. In one example, the subject has COVID-19, for example, severe COVID-19. In particular, severe COVID-19 often results in ARDS. The methods of the present disclosure can be used to treat or prevent ARDS in a subject suffering from severe COVID-19.

### Acute Respiratory Distress Syndrome (ARDS)

The present disclosure provides, for example, methods of treating, preventing and/or delaying progression of ARDS in a subject.

ARDS is a life-threatening condition characterized by bilateral pulmonary infiltrates, severe hypoxemia, and disruption of the alveolar-capillary membrane barrier (i.e., pulmonary vascular leak), leading to non-cardiogenic pulmonary edema. There is currently no effective pharmacological therapy.

Infectious etiologies, including influenza and coronavirus infection, are leading causes of ARDS. Accordingly, in one example of the present disclosure, the ARDS is associated with an influenza or a coronavirus infection. For example, the ARDS is associated with influenza. In another example, the ARDS is associated with a coronavirus infection, such as a SARS-COV infection. In one example, the ARDS is associated with a SARS-COV-2 infection.

ARDS is classified according to the Berlin Definition, which includes:
(1) presentation within 1 week of clinical insult or onset of respiratory symptoms;
(2) acute hypoxemic respiratory failure, as determined by a PaO2/FiO2 ratio of 300 mmHg or less on at least 5 cm of continuous positive airway pressure (CPAP) or positive end expiratory pressure (PEEP), where PaO2 is the partial pressure of oxygen in arterial blood and the FiO2 is the fraction of inspired oxygen;
(3) bilateral opacities on lung radiographs not fully explained by effusions, consolidation, or atelectasis; and
(4) edema/respiratory failure not fully explained by cardiac failure or fluid overload.

In one example, the subject has or suffers from ARDS (i.e., the subject satisfies the Berlin definition of ARDS). For example, the subject is in need of treatment (i.e., in need thereof).

In one example, the subject has or suffers from a symptom associated with ARDS. Symptoms associated with ARDS and methods of identifying subjects at risk of developing ARDS will be apparent to the skilled person and/or are described herein. For example, the subject has one or more or all of the following symptoms:
(a) a respiratory frequency of greater than 30 breaths per minute;
(b) an oxygen saturation (SpO₂) of 93% or less on room air;
(c) a ratio of arterial partial pressure of oxygen to fraction of inspired oxygen (PaO₂/FiO₂) of less than 300 mmHg;
(d) a SpO₂/FiO₂ ratio of less than 218; and
(e) radiographic lung infiltrates in an amount of greater than 50%.

Currently, ARDS is classified as mild, moderate or severe with an associated increased mortality. The severity of ARDS can be categorized according to the Berlin definition as follows:
(a) Mild ARDS: PaO₂/FiO₂ of 200-300 mmHg on at least 5 cm CPAP or PEEP;
(b) Moderate ARDS: PaO₂/FiO₂ of 100-200 mmHg on at least 5 cm PEEP; and
(c) Severe ARDS: PaO₂/FiO₂ of less than or equal to 100 mmHg on at least 5 cm PEEP.

In one example, the ARDS is mild ARDS. In another example, the ARDS is moderate ARDS. In a further example, the ARDS is severe ARDS.

The methods can, in addition to treatment of existing ARDS, be used to prevent the onset of ARDS. Thus, in one example, the subject does not have ARDS.

### Pharmaceutical Compositions

Proteins and antibodies of the disclosure (syn. active ingredients) are useful for formulations into a pharmaceutical composition for parenteral, topical, oral, or local administration, aerosol administration, or transdermal administration, for prophylactic or for therapeutic treatment. The pharmaceutical compositions can be administered in a variety of unit dosage forms depending upon the method of administration. For example, unit dosage forms suitable for oral administration include powder, tablets, pills, capsules and lozenges.

The pharmaceutical compositions of this disclosure are useful for parenteral administration, such as intravenous administration or subcutaneous administration or administration into a body cavity or lumen of an organ or joint. The compositions for administration will commonly comprise a solution of the protein or the antibody of the disclosure dissolved in a pharmaceutically acceptable carrier, such as an aqueous carrier. A variety of aqueous carriers can be used, e.g., buffered saline and the like. The compositions may contain pharmaceutically acceptable carriers as required to approximate physiological conditions such as pH adjusting and buffering agents, toxicity adjusting agents and the like, for example, sodium acetate, sodium chloride, potassium chloride, calcium chloride, sodium lactate and the like. The concentration of the protein or the antibody of the present disclosure in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

The protein or the antibody of the disclosure can be formulated for parenteral administration, e.g., formulated for injection via the intravenous, intramuscular, subcutaneous, transdermal, or other such routes, including peristaltic administration and direct instillation into a tumor or disease site (intracavity administration). The preparation of an aqueous composition that contains the compounds of the present disclosure as an active ingredient will be known to those of skill in the art.

Suitable pharmaceutical compositions in accordance with the disclosure will generally include an amount of the protein or the antibody of the present disclosure admixed with an acceptable pharmaceutical carrier, such as a sterile aqueous solution, to give a range of final concentrations, depending on the intended use. The techniques of preparation are generally known in the art as exemplified by Remington's Pharmaceutical Sciences, 16th Ed. Mack Publishing Company, 1980.

### Dosage and Administration

Upon formulation, proteins and antibodies of the present disclosure will be administered in a manner compatible with the dosage formulation and in such amount as is therapeutically/prophylactically effective.

The dosage should not be so large as to cause adverse side effects. Generally, the dosage will vary with the age, condition, sex and extent of the disease in the patient and can be determined by one of skill in the art. The dosage can be adjusted by the individual physician in the event of any complication.

Dosage can vary from about 0.1 mg/kg to about 300 mg/kg, e.g., from about 0.2 mg/kg to about 200 mg/kg, such as, from about 0.5 mg/kg to about 20 mg/kg, in one or more dose administrations daily, for one or several days.

In some examples, the protein or antibody is administered at an initial (or loading) dose which is higher than subsequent (maintenance doses). For example, the protein or antibody is administered at an initial dose of between about 1 mg/kg to about 30 mg/kg. The protein or antibody is then administered at a maintenance dose of between about 0.0001 mg/kg to about 1 mg/kg. The maintenance doses may be administered every 7-35 days, such as, every 14 or 21 or 28 days.

In some examples, a dose escalation regime is used, in which a protein or antibody is initially administered at a lower dose than used in subsequent doses. This dosage regime is useful in the case of subject's initially suffering adverse events

In the case of a subject that is not adequately responding to treatment, multiple doses in a week may be administered. Alternatively, or in addition, increasing doses may be administered.

A subject may be retreated with the protein and/or antibody, by being given more than one exposure or set of doses, such as at least about two exposures of the protein and/or antibody, for example, from about 2 to 60 exposures, and more particularly about 2 to 40 exposures, most particularly, about 2 to 20 exposures.

In another example, any retreatment may be given at defined intervals. For example, subsequent exposures may be administered at various intervals, such as, for example, about 24-28 weeks or 48-56 weeks or longer. For example, such exposures are administered at intervals each of about 24-26 weeks or about 38-42 weeks, or about 50-54 weeks.

### Kits

Another example of the disclosure provides kits containing a protein of the present disclosure useful for treating, preventing and/or delaying progression of a respiratory viral infection as described above.

In one example, the kit comprises (a) a container comprising a protein optionally in a delivery system and/or a pharmaceutically acceptable carrier or diluent; and (b) a package insert with instructions for treating, preventing and/or delaying progression of a respiratory viral infection (e.g., SARS-COV-2 infection, COVID-19 and/or ARDS) in a subject.

In accordance with this example of the disclosure, the package insert is on or associated with the container. Suitable containers include, for example, bottles, vials, syringes, etc. The containers may be formed from a variety of materials such as glass or plastic. The container holds or contains a composition that is effective for a disease or disorder of the disclosure and may have a sterile access port (for example, the container may be an intravenous solution bag or a vial having a stopper pierceable by a hypodermic injection needle). At least one active agent in the composition is the protein. The label or package insert indicates that the composition is used for treating a subject eligible for treatment, e.g., one having or predisposed to developing a respiratory viral infection (e.g., SARS-COV-2 infection, COVID-19 or ARDS) with specific guidance regarding dosing amounts and intervals of treatment and any other medicament being provided. The kit may further comprise an additional container comprising a pharmaceutically acceptable diluent buffer, such as bacteriostatic water for injection (BWFI), phosphate-buffered saline, Ringer's solution, and/or dextrose solution. The kit may further include other materials desirable from a commercial and user standpoint, including other buffers, diluents, filters, needles, and syringes.

Another example of the disclosure provides kits containing proteins or antibodies of the disclosure for use in any method described herein (e.g., detecting an antigen having a conformation sufficient to induce an immune response against SARS-COV-2).

In one example, the kit or panel of proteins or antibodies for detecting an antigen having a conformation sufficient to induce an immune response against SARS-COV-2 comprises one or more proteins or antibodies described herein. In one example, the antigen is a protein, a peptide, an attenuated virus or a virus-like particle.

The present disclosure includes the following non-limiting Examples.

### EXAMPLES

### Example 1: Monoclonal Antibody Development

Monoclonal antibodies against SARS-COV-2 S protein RBD were produced by immunising Balb/c mice with 10 μg of HEK293 SARS-COV2 S protein (S1 Subunit, His Tag (Sino Biological) mixed with an equal volume of complete Fruends' adjuvant. Popliteal lymph nodes were harvested from Balb/c immunized mice and the lymphocytes were fused and selected as previously described Pietrzykowski et al 2002 to generate hybrdoma cell lines. The hybridoma cells were cloned and screened using 4 μg/ml SARS-COV2 Spike Protein (S1 Subunit, RBD His Tag expressed in Baculovirus (Sino Biological) using a solid phase ELISA. Antibody binding was detected a HRP Goat anti Mouse IgG (gamma) followed by chromatographic conversion. Confirmation of the reactivity was achieved by subsequent screening of the antibody secreting hybridomas with a 2019 nCOV S protein RBD, Fc Tag (Acro Biosystems) using solid phase ELISA. Isotype determinations (Heavy/light chains) for each cloned monoclonal antibody was determined using a Roche IsoStrip Mouse Monoclonal Antibody Isotyping Kit (Merck 11493027001) as per manufacturer's instructions.

A total of 34 anti-SARS-COV-2 S protein monoclonal antibodies were identified by ELISA. Monoclonal antibody clones 30B8 and 6G6 were used to further determine ability to bind SARS-COV-2 S protein RBD expressed in yeast and E. coli (which have a different glycosylation profile) on solid phase ELISA. Monoclonal antibodies 30B8 and 6G6 demonstrated no to weak reactivity to E. coli expressed SARS-COV-2 S protein RBD, and modest reactivity to yeast expressed SARS-COV-2 S protein RBD.

The results suggest that E. coli expressed SARS-COV-2 S protein RBD do not have a protein conformation reflective of mammalian expression systems, and yeast expressed which bind to monoclonal antibodies 30B8 and 6G6 but may not be in a trimeric form since the 30B8 capture/detection ELISA was negative while the 30B8 capture and 6G6 detection ELISA was positive.

### Example 2: In Vitro Neutralisation Assays of SARS-COV-2

To determine the neutralisation capabilities of the 34 anti-SARS-COV-2 monoclonal antibodies generated and identified from the method as described in Example 1, three different virus neutralisation assays were performed: (1) Vero microneutralisation; (2) pseudovirus neutralisation; and (3) sVNT (surrogate virus neutralisation test). The results are summarised in Table 1 below.

The pseudovirus assay is used to demonstrate ability of the anti-SARS-COV-2 monoclonal antibody to prevent viral entry into a cell, while the Vero neutralisation assay (described above) is used to demonstrate ability of the anti-SARS-COV-2 monoclonal antibody to neutralize viral entry into a cell and also to prevent viral replication. The neutralising screening results demonstrated that a number of the anti-SARS-COV-2 monoclonal antibody candidates were able to sterically hinder RBD binding to ACE2 in the sVNT assay but were unable to inhibit pseudovirus entry and/or wild-type virus entry and replication in the other assays. The anti-SARS-COV-2 monoclonal antibody candidates showed neutralising capability in at least one or more of the three assays (Table 1).

TABLE 1
Neutralisation evaluation of monoclonal antibodies for SARS COV-2

| | | Vero Cell | | SVNT |
|---|---|---|---|---|
| Clone Parental | | Virus | | (% |
| Line | Clone ID | inhibition | Pseudovirus ª | Inhibition) |
| COV185.2C11 | 6G6 | + | 55000 | 90 |
| COV185.4E2 | 19G2 | + | NT | 30 |
| COV185.6E6 | 27C8 | − | 30000 | 55 |
| COV185.8E6 | 28B2 | − | 10000 | 60 |
| COV185.9F6 | 30B8 | + | 15000 | 45 |
| COV185.8E10 | 39E7 | + | NT | 40 |
| TORA1843F4 | 33E10 | − | NT | 30 |
| COV185.6E6 | 28B6 | − | 10000 | 83 |

NT Not tested
ª The pseudovirus number represents the area under the neutralisation curve. The higher the number the greater the neutralisation.

### Vero Microneutralisation Assays

Wild type SARS-COV-2 virus is capable of undergoing rounds of replication in Vero cells. Accordingly, the ability of the 34 anti-SARS-COV-2 monoclonal antibodies, generated and identified from the method as described in Example 1, to prevent viral replication was tested.

Serial two-fold dilutions of anti-SARS-COV-2 monoclonal antibody were incubated with 100 TCID₅₀ of SARS-COV-2 isolate CoV/Australia/VIVC01/2020 (Caly et al 2020) for 1 hour and residual infectivity with and without pre-incubation anti-SARS-COV-2 monoclonal antibodies was assessed in Vero cells 5 days post infection. The neutralising antibody titre was calculated using the Reed/Muench method as previously described (Houser et al., 2016; Subbarao et al 2004).

### Pseudovirus Assays

To determine the ability of anti-SARS-COV-2 monoclonal antibody to bind the receptor binding domain of the spike protein on the pseudovirus and thereby preventing viral entry into a cell, pseudovirus assays were conducted. HIV reporter virus pseudotyped with SARS-2-Spike protein was produced by Lipofetamine co-transfection of 8 μg of different SARS-2-COV-2 spike plasmids together with 16 μg of the viral backbone plasmid (pDR-NL Δenv FLUC) into 8.10⁶ HEK-293T cells. Viral stocks were produced from the reference Wuhan sequence as well as circulating spike mutants to analyse the antibody neutralisation. Table 2 shows the SARS-COV-2 spike selected for the panel.

TABLE 2
SARS-COV-2 spike protein

| SARS-COV-2 Spike | Plasmid and aa modification |
|---|---|
| Wuhan Δ418 | pcDNA 3.1 (Invitrogen) with original |
| | SARS-COV-2 Spike corresponding to the |
| | virus isolated in Wuhan cloned in between |
| | EcoRI and BamHI sites. The last 18 amino |
| | acids (c-terminus) were deleted to remove |
| | the endoplasmic reticulum retention |
| | signal. |
| D614G Δ18 | pcDNA 3.1 (Invitrogen) SARS-COV-2 |
| | Spike with a single mutation in the aa 614 |
| | from D to G. The last 18 amino acids (c- |
| | terminus) were deleted to remove the |
| | endoplasmic reticulum retention signal. |
| S477N-D614G Δ18 | pcDNA 3.1 (Invitrogen) SARS-COV-2 |
| | Spike with the double mutation, position |
| | 614 from D to G and position 477 from S |
| | to N. The last 18 amino acids (c-terminus) |
| | were deleted to remove the endoplasmic |
| | reticulum retention signal. |
| G485R-D614G Δ18 | pcDNA 3.1 (Invitrogen) SARS-COV-2 |
| | Spike with a mutation in the aa 614 from |
| | D to G and position 485 from G to R. The |
| | last 18 amino acids (c-terminus) were |
| | deleted to remove the endoplasmic |
| | reticulum retention signal. |
| N501Y-D614G Δ18 | pcDNA 3.1 (Invitrogen) SARS-COV-2 |
| | Spike with a mutation in the aa 614 from |
| | D to G and position 501 from N to Y. The |
| | last 18 amino acids (c-terminus) were |
| | deleted to remove the endoplasmic |
| | reticulum retention signal. |

Pseudovirus were harvested 48 hrs post transfection and clarified by filtration through a 45 μm filter, aliquoted and stored at −80° C. Virus stock titres, reported as Relative Luciferase Units infectious dose (RLU), were calculated by limiting dilution infections in Hela-hACE2 cells, the same target cells utilised in the neutralisation assays, measuring luciferase activity as a read-out for viral infection.

To test neutralisation activity of the monoclonal antibodies generated in Example 1, 1 in 5 serial dilutions of antibody (DMEM wo FBS) were mixed with 100.000 RLU of virus in a volume of 150 μl. The virus-antibody mix was incubated for 1 h at 37° C. to allow for neutralisation. After incubation, the virus-antibody mix was added to 1.104 Hela-ACE2 cells seeded on 96 well-plate (total volume cell-virus-Antibody 250 μl) and spinoculated for 2 h at 1500 g. Cells were further incubated at 37° C., 5% CO2 for 72 h.

At the end of the incubation period, 150 μl media was removed from the cells leaving 100 μl residual media on the plate, and an equal volume of Britelite Plus reagent (Luciferase enzyme substrate Cat #6066769) was added for 2 mins to allow cell lysis and enzymatic conversion to a luminescence product. To read virus infectivity, 100 μl of the cell lysate was transferred to a black-walled plate and luminescence signal was measured on a FLUOStar microplate reader.

Antibody candidates were tested in duplicate and the percentage of neutralisation was determined by calculating the difference in average RLU between virus control (virus incubated with dilutions of the Hybridoma Media) and test wells, dividing this result by the difference in average RLU between virus control and uninfected cell control wells (background), and multiplying by 100.

Neutralising antibody titers are expressed as the monoclonal antibody concentration (μg/ml) required for 50% reduction of relative luminescence units (RLU) compared to the level in the virus control wells. Graphs plotting the Percent Neutralisation vs IgG concentration were created and a Non-linear regression model (log [Ab conc]. vs response/3 parameters curve) were used to best fit the neutralisation curve. From each fitted curve (r2 coefficient over 0.7), the 50% inhibitory concentration (IC50) values were estimated. The IC50 value denotes the antibody concentration that corresponds with 50% neutralisation in each fitted titration curve.

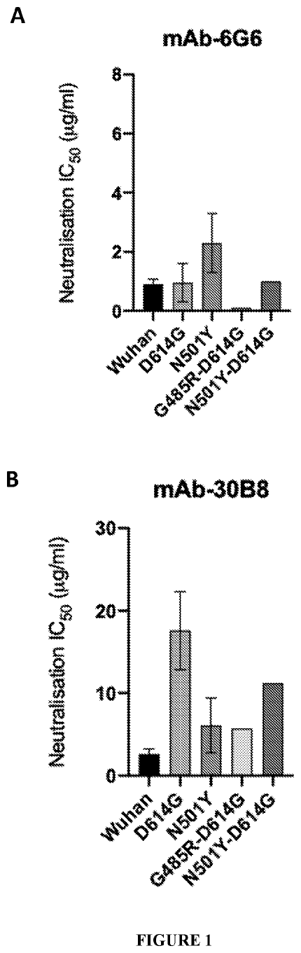

FIG. 1 shows the neutralising capabilities of monoclonal antibodies 6G6 and 30B8 against wild type SARS-COV-2 spike protein (Wuhan), single mutant SARS-COV-2 spike protein (D614G, N501Y) and double mutant SARS-COV-2 spike protein (G485R-D614G, N501Y-D614G) determined by pseudovirus assays. The SARS-COV-2 spike proteins of FIG. 1 are those described in Table 2 (above). Monoclonal antibody 6G6 demonstrated greater neutralising capability with lower concentrations of the antibody required to neutralize wild type and all mutant SARS-COV-2 spike proteins compared to antibody 30B8. A greater concentration of monoclonal antibody 30B8 was required to neutralize mutant N501Y SARS-COV-2 spike protein compared to neutralisation of wild type SARS-COV-2 spike protein (Wuhan). A lower concentration of monoclonal antibody 6G6 was required to neutralize double mutant SARS-COV-2 spike protein (N501Y-D614G) compared to single mutant N501Y SARS-COV-2 spike protein.

sVNT (Surrogate Virus Neutralisation Test)

The sVNT is a surrogate neutralisation assay as it involves mixing the anti-SARS-CoV-2 monoclonal antibody with a tagged RBD protein to determine whether the anti-SARS-COV-2 monoclonal antibody can inhibit binding of the spike protein RBD to recombinant ACE2 protein bound on a plastic plate. The sVNT is a useful assay for screening monoclonal antibodies for their ability to bind to RBD.

The hACE2 protein (GenScript) was coated at 100 ng/well in 100 mM carbonate-bicarbonate coating buffer (pH 9.6). HRP-conjugated SARS-COV-2 HRP-conjugated SARS-COV-RBD (GenScript) was added to the hACE2 coated plate at different concentration in OptEIA assay diluent (BD) for 1 h at room temperature. Unbound HRP conjugated antigens were removed by washing with phosphate buffered saline, 0.05% tween-20 (PBST). Colorimetric signal was developed on the enzymatic reaction of HRP with chromogenic substrate, 3,3′,5,5′-tetramethylbenzidine (TMB) (Invitrogen). Equal volume of TMB stop solution (KPL) was added to stop the reaction, and the absorbance reading at 450 nm and 570 nm were acquired using Cytation 5 microplate reader (BioTek). For the surrogate neutralisation test (sVNT), 6 ng of HRP-RBD (from either virus) was pre-incubated with monoclonal antibodies at dilution of 1:20 for 1 h at 37° C., followed by hACE2 incubation for 1 h at room temperature. Inhibition (%)=(1-Sample OD value/Negative Control OD value)×100.

### Example 3: Hybridoma Sequence Analysis

The antibody sequencing protocol involves isolating the mRNA from hybridoma cells followed by cDNA synthesis and PCR amplification of heavy- and light-chain variable region genes. The genes were then cloned into the pGEN-T easy vector. The clones were screened by PCR and positive clones are sequenced (Sanger sequencing). Analysis of the V_{H} and VL gene sequences was performed using the IMGT/V-Quest program, (The International Immunogenetics Information System; http://www.imgt.org/IMGT_vquest/vquest).

### Example 4: Epitope Binding

Antibody 30B8 and two publicly available anti-SARS-COV-2 antibodies B38 (disclosed in Wu et al., Science 368:1274-128, 2020) and AS35 (Cat. No. SPD-S68 Acrobiosystems) were tested for binding against a pool of 13 biotinylated peptides (including one negative control). Each peptide was 15 amino acids in length, with each peptide having a 4 amino acid overlap.

Neither B38 nor AS35 had an OD response above background for any of the peptides analysed. Antibody 30B8 had an OD response above background and only cross-reacted against peptide 40 (SEQ ID NO: 51; SGSGAGSTPCNGVEGENCY).

Mapping indicated that antibody 30B8 bound the non-linear epitope AGNCY corresponding to amino acid residues 5, 6, 17, 18 and 19 of peptide 40.

TABLE 3
Table of sequences

| SEQ ID | Molecule | |
|---|---|---|
| NO | type | Residues |
| 1 | Protein | DVQLQESGPGLVKPSQSLSLTCTVTGYSITSDYAWNWIRQFP |
| | | GNKLEWMGYISYSGSTSYNPSLKSRISITRDTSKNQFFLQLKS |
| | | VTTEDAATYYCARGDGYFYAMDYWGQGTSVTVSS |
| 2 | Protein | DIVMTQSPSSLSVSAGEKVTMSCKSSQSLLNSGNQKSYLAWY |
| | | QQKPGQPPKLLIYGASTRESGVPDRFTGSGSGTDFTLTISSVQ |
| | | AEDLAVYSCQNDHSYPPTFGAGTKLDLK |
| 3 | Protein | EVQLQQSGAELVKPGASVKLSCTASDFNIKDTYMHWVKQRP |
| | | EQGLEWIGRIDPASDNSKYDPKFQGKATITPDTSSNTAYLQLS |
| | | SLTSEDTAVYYCASSYYDYDGSWFAYWGQGTLVTVSA |
| 4 | Protein | ENVLTQSPAIMSASPGEKVTMTCSARSSVSYMHWYQQKSST |
| | | SPKVWIYDTSKLASGVPGRFSGSGSGNSYSLTISSMEAEDVAT |
| | | YYCFQGSGYPLTFGSGTKLEIK |
| 5 | Protein | EVQLQQSGAELVKPGASVKLSCTASGFNIKDTYIHWVKQRPE |
| | | QGLEWIGRIDPANGNTKYDPKFQGKATITADTSSNTAYLQLS |
| | | SLTSEDTAVCYCAGSLYDYDGAPFAYWGQGTLVTVSA |
| 6 | Protein | SIVMTQTPKFLLVSAGDRVTITCKASQSVTNDVTWYQQKPG |
| | | QSPKLLIYYASNRYTGVPDRFTGSGYGTDFTFTISTVQAEDLA |
| | | VYFCQQDYSSPWTFGGGTKLEIK |
| 7 | Protein | EVQLQQSGAELVKPGASVKLSCTVSGFNIKDTYMHWVKQRP |
| | | EQGLEWIGRIDPANGNTKYDPKFQGKATITADTSSNTAYLQL |
| | | SSLTSEDTAVYYCASSYYDYDGSWFAYWGQGTLVTVSA |
| 8 | Protein | ENVLTQSPAIMSASPGEKVTMTCSASSSVSYMHWYQQKSSTS |
| | | PKIWIYDTSKLASGVPGRFSGSGSGNSYSLTISSMEAEDVATY |
| | | YCFQGSGYPLTFGSGTRLEIK |
| 9 | Protein | QVQLQQSGPQLVRPGASVKISCKASGYSFTNNWMHWVKQR |
| | | PGQGLEWIGMIDPSDSETRLNQKFKDKATLTVDKSSSTAYMQ |
| | | LSSPTSEDSAVYYCARVNWEIFDYWGQGTTLTVSS |
| 10 | Protein | DIVMTQAAPSVPVTPGESVSISCRSSKSLLNRNGNTYLYWFL |
| | | QRPGQSPQLLIYRMSNLASGVPDRFSGSGSGTAFTLRISRVEA |
| | | EDVGVYYCMQHLEYPLTFGAGTKLELK |
| 11 | DNA | gatgtgcagcttcaggagtcgggacctggcctggtgaaaccttctcagtctctgtccctcacctgc |
| | | actgtcactggctactcaatcaccagtgattatgcctggaactggatccggcagtttccaggaaac |
| | | aaactggagtggatgggctacataagctacagtggtagcactagctacaacccatctctcaaaag |
| | | tcgaatctctatcactcgagacacatccaagaaccagttcttcctgcagttgaaatctgtgactactg |
| | | aggacgcagccacatattactgtgcaagaggcgatggttatttctatgctatggactactggggtc |
| | | aaggaacctcagtcaccgtctcctca |
| 12 | DNA | gacattgtgatgacacagtctccatcctccctgagtgtgtcagctggagagaaggtcactatgagc |
| | | tgcaagtccagtcagagtctgttaaacagtggaaatcaaaagagctacttggcctggtaccagca |
| | | gaaaccagggcagcctcctaaactgttgatctacggggcatccactagggaatctggggtccct |
| | | gatcgcttcacaggcagtggatctggaaccgatttcactcttaccatcagcagtgtgcaggctgaa |
| | | gacctggcagtttattcctgtcagaatgatcatagttatcctcccacgttcggtgctgggaccaagc |
| | | tggacctgaaa |
| 13 | DNA | gaggttcagctgcagcagtctggggcagagcttgtgaagccaggggcctcagtcaagttgtcct |
| | | gcacagcttctgacttcaacattaaagacacctatatgcactgggtgaagcagaggcctgaacag |
| | | ggcctggagtggattggaaggattgatcctgcgagtgataatagtaaatatgacccgaagttcca |
| | | gggcaaggccactataacaccagacacatcctccaacacagcctacctgcagctcagcagcct |
| | | gacatctgaggacactgccgtctattactgtgcctcatcctactatgattacgacggttcctggtttg |
| | | cttactggggccaagggactctggtcactgtctctgca |
| 14 | DNA | gaaaatgttctcacccagtctccagcaatcatgtctgcatctccaggggaaaaggtcaccatgacc |
| | | tgcagtgccaggtcaagtgtaagttacatgcactggtaccagcagaagtcaagcacctcccccaa |
| | | agtctggatttatgacacatccaaactggcttctggggtcccaggtcgcttcagtggcagtgggtct |
| | | ggaaactcttactctctcacgatcagcagcatggaggctgaagatgttgccacttattactgttttca |
| | | ggggagtgggtacccactcacgttcggctcggggacaaagttggaaataaaa |
| 15 | DNA | gaggttcagctgcagcagtctggggcagagcttgtgaagccaggggcctcagtcaagttgtcct |
| | | gcacagcttctggcttcaacattaaagacacctatatacactgggtgaagcagaggcctgaacag |
| | | ggcctggagtggattggaaggattgatcctgcgaatggtaatactaaatatgacccgaagttcca |
| | | gggcaaggccaccataacagcagacacatcctccaacacagcctacctgcagctcagcagcct |
| | | gacatctgaggacactgccgtctgttactgtgctgggtccctctatgattacgacggggccccgttt |
| | | gcttactggggccaagggactctggtcactgtctctgca |
| 16 | DNA | agtattgtgatgacccagactcccaaattcctgcttgtatcagcaggagacagggttaccataacct |
| | | gcaaggccagtcagagtgtgactaatgatgtaacttggtaccaacagaagccagggcagtctcct |
| | | aaactgctgatatactatgcatccaatcgctacactggagtccctgatcgcttcactggcagtggat |
| | | atgggacggatttcactttcaccatcagcactgtgcaggctgaagacctggcagtttatttctgtca |
| | | gcaggattatagctctccgtggacgttcggtggaggcaccaagctggaaatcaaa |
| 17 | DNA | gaggttcagctgcagcagtctggggcagagcttgtgaagccaggggcctcagtcaagttgtcct |
| | | gcacagtttctggcttcaacattaaagacacctatatgcactgggtgaagcagaggcctgaacag |
| | | ggcctggagtggattggaaggattgatcctgcgaatggtaatactaaatatgacccgaagttcca |
| | | gggcaaggccactataacagcagacacatcctccaacacagcctacctgcaactcagcagcct |
| | | gacatctgaggacactgccgtctattactgtgcctcatcctactatgattacgacggttcctggtttg |
| | | cttactggggccaagggactctggtcactgtctctgca |
| 18 | DNA | gaaaatgttctcacccagtctccagcaatcatgtctgcatctccaggggaaaaggtcaccatgacc |
| | | tgcagtgccagctcaagtgtaagttacatgcactggtaccagcagaagtcaagcacctcccccaa |
| | | aatctggatttatgacacatccaaactggcttctggagtcccaggtcgcttcagtggcagtgggtct |
| | | ggaaactcttactctctcacgatcagcagcatggaggctgaagatgttgccacttattactgttttca |
| | | ggggagtgggtacccactcacgttcggctcggggacaaggttggaaataaaa |
| 19 | DNA | caggtgcaactacaacagtctgggcctcagctggttaggcctggggcttcagtgaagatatcctg |
| | | caaggcttctggttactcattcaccaataactggatgcactgggtgaagcagaggcctggacaag |
| | | gtcttgagtggattggcatgattgatccttccgatagtgaaactaggttaaatcagaaattcaagga |
| | | caaggccacattgactgtagacaaatcctccagcacagcctacatgcaactcagcagcccgaca |
| | | tctgaggactctgcggtctattactgtgcaagggttaactgggaaatctttgactactggggccaag |
| | | gcaccactctcacagtctcctca |
| 20 | DNA | gatattgtgatgactcaggctgcaccctctgtacctgtcactcctggagagtcagtatccatctcctg |
| | | caggtctagtaagagtctcctgaatagaaatggcaacacttacttgtattggttcctgcagaggcca |
| | | ggccagtctcctcagctcctgatatatcggatgtccaaccttgcctcaggagtcccagacaggttc |
| | | agtggcagtgggtcaggaactgctttcacactgagaatcagtagagtggaggctgaggatgtgg |
| | | gtgtttattactgtatgcaacatctagaatatccgctcacgttcggtgctgggaccaagctggagct |
| | | gaaa |
| 21 | Protein | GYSITSDYA |
| 22 | Protein | ISYSGST |
| 23 | Protein | ARGDGYFYAMDY |
| 24 | Protein | QSLLNSGNQKSY |
| 25 | Protein | GAS |
| 26 | Protein | QNDHSYPPT |
| 27 | Protein | DFNIKDTY |
| 28 | Protein | IDPASDNS |
| 29 | Protein | GFNIKDTY |
| 30 | Protein | IDPANGNT |
| 31 | Protein | AGSLYDYDGAPFAY |
| 32 | Protein | QSVTND |
| 33 | Protein | YAS |
| 34 | Protein | QQDYSSPWT |
| 35 | Protein | ASSYYDYDGSWFAY |
| 36 | Protein | SSVSY |
| 37 | Protein | DTS |
| 38 | Protein | FQGSGYPLT |
| 39 | Protein | GYSFTNNW |
| 40 | Protein | IDPSDSET |
| 41 | Protein | ARVNWEIFDY |
| 42 | Protein | KSLLNRNGNTY |
| 43 | Protein | RMS |
| 44 | Protein | MQHLEYPLT |
| 45 | Protein | EVQLQQSGAELVKPGASVKLSCTXSXFNIKDTYXHWVKQRP |
| | | EQGLEWIGRIDPAXXNXKYDPKFQGKATITXDTSSNTAYLQL |
| | | SSLTSEDTAVXYCAXSXYDYDGXXFAYWGQGTLVTVSA |
| 46 | Protein | ENVLTQSPAIMSASPGEKVTMTCSAXSSVSYMHWYQQKSST |
| | | SPKXWIYDTSKLASGVPGRFSGSGSGNSYSLTISSMEAEDVAT |
| | | YYCFQGSGYPLTFGSGTXLEIK |
| 47 | Protein | XFNIKDTY |
| 48 | Protein | IDPAXXNX |
| 49 | Protein | AXSXYDYDGXXFAY |
| 50 | Protein | MFVFLVLLPLVSSQCVNLTTRTQLPPAYTNSFTRGVYYPDKV |
| | | FRSSVLHSTQDLFLPFFSNVTWFHAIHVSGTNGTKRFDNPVLP |
| | | FNDGVYFASTEKSNIIRGWIFGTTLDSKTQSLLIVNNATNVVI |
| | | KVCEFQFCNDPFLGVYYHKNNKSWMESEFRVYSSANNCTFE |
| | | YVSQPFLMDLEGKQGNFKNLREFVFKNIDGYFKIYSKHTPINL |
| | | VRDLPQGFSALEPLVDLPIGINITRFQTLLALHRSYLTPGDSSS |
| | | GWTAGAAAYYVGYLQPRTFLLKYNENGTITDAVDCALDPLS |
| | | ETKCTLKSFTVEKGIYQTSNFRVQPTESIVRFPNITNLCPFGEV |
| | | FNATRFASVYAWNRKRISNCVADYSVLYNSASFSTFKCYGV |
| | | SPTKLNDLCFTNVYADSFVIRGDEVRQIAPGQTGKIADYNYK |
| | | LPDDFTGCVIAWNSNNLDSKVGGNYNYLYRLFRKSNLKPFE |
| | | RDISTEIYQAGSTPCNGVEGFNCYFPLQSYGFQPTNGVGYQP |
| | | YRVVVLSFELLHAPATVCGPKKSTNLVKNKCVNFNFNGLTG |
| | | TGVLTESNKKFLPFQQFGRDIADTTDAVRDPQTLEILDITPCSF |
| | | GGVSVITPGTNTSNQVAVLYQDVNCTEVPVAIHADQLTPTW |
| | | RVYSTGSNVFQTRAGCLIGAEHVNNSYECDIPIGAGICASYQT |
| | | QTNSPRRARSVASQSIIAYTMSLGAENSVAYSNNSIAIPTNFTI |
| | | SVTTEILPVSMTKTSVDCTMYICGDSTECSNLLLQYGSFCTQL |
| | | NRALTGIAVEQDKNTQEVFAQVKQIYKTPPIKDFGGFNFSQIL |
| | | PDPSKPSKRSFIEDLLFNKVTLADAGFIKQYGDCLGDIAARDL |
| | | ICAQKFNGLTVLPPLLTDEMIAQYTSALLAGTITSGWTFGAG |
| | | AALQIPFAMQMAYRFNGIGVTQNVLYENQKLIANQFNSAIGK |
| | | IQDSLSSTASALGKLQDVVNQNAQALNTLVKQLSSNFGAISS |
| | | VLNDILSRLDKVEAEVQIDRLITGRLQSLQTYVTQQLIRAAEI |
| | | RASANLAATKMSECVLGQSKRVDFCGKGYHLMSFPQSAPHG |
| | | VVFLHVTYVPAQEKNFTTAPAICHDGKAHFPREGVFVSNGTH |
| | | WFVTQRNFYEPQIITTDNTFVSGNCDVVIGIVNNTVYDPLQPE |
| | | LDSFKEELDKYFKNHTSPDVDLGDISGINASVVNIQKEIDRLN |
| | | EVAKNLNESLIDLQELGKYEQYIKWPWYIWLGFIAGLIAIVM |
| | | VTIMLCCMTSCCSCLKGCCSCGSCCKFDEDDSEPVLKGVKLH |
| | | YT |
| 51 | Protein | SGSGAGSTPCNGVEGENCY |

1. A protein comprising an antibody variable region, wherein the antibody variable region binds to or specifically binds to a severe acute respiratory syndrome coronavirus 2 (SARS-COV-2) spike(S) protein receptor binding domain (RBD) and neutralises binding of the S protein to angiotensin-converting enzyme 2 (ACE2), and wherein the antibody variable region competitively inhibits binding of any one of the following antibodies to the SARS-COV-2 S protein RBD:
(a) an antibody comprising a heavy chain variable region (V_{H}) comprising a sequence set forth in SEQ ID NO: 1 and a light chain variable region (V_{L}) comprising a sequence set forth in SEQ ID NO: 2;
(b) an antibody comprising a V_{H} comprising a sequence set forth in SEQ ID NO: 3 and a V_{L} comprising a sequence set forth in SEQ ID NO: 4;
(c) an antibody comprising a V_{H} comprising a sequence set forth in SEQ ID NO: 5 and a V_{L} comprising a sequence set forth in SEQ ID NO: 6;
(d) an antibody comprising a V_{H} comprising a sequence set forth in SEQ ID NO: 7 and a V_{L} comprising a sequence set forth in SEQ ID NO: 8; and
(e) an antibody comprising a V_{H} comprising a sequence set forth in SEQ ID NO: 9 and a V_{L} comprising a sequence set forth in SEQ ID NO: 10.

2. The protein of claim 1, wherein the protein neutralises binding of the SARS-COV-2 S protein RBD to ACE2 transfected VeroE6 cells with a half-maximal inhibitory concentration (IC₅₀) of at least about 5 μg/ml.

3. The protein of claim 1, wherein the antibody variable region:
(i) binds specifically to a conformational epitope in the SARS-COV-2 S protein RBD, the RBD comprising amino acid residues 5, 6, 17, 18 and 19 of a sequence set forth in SEQ ID NO: 51;
(ii) binds to a mutant S protein, wherein the mutant S protein comprises:
(a) D to G mutation at residue corresponding to nucleotide 614 of SEQ ID NO: 50; and/or
(b) N to Y mutation at residue corresponding to nucleotide 501 of SEQ ID NO: 50; and/or
(c) S to N mutation at residue corresponding to nucleotide 477 of SEQ ID NO: 50; and/or
(d) G to R mutation at residue corresponding to nucleotide 485 of SEQ ID NO: 50; and/or
(iii) binds specifically to the same epitope in the SARS-COV-2 S protein RBD as that bound by any one of the following antibodies:
(a) an antibody comprising a V_{H} comprising a sequence set forth in SEQ ID NO: 1 and a V_{L} comprising a sequence set forth in SEQ ID NO: 2;
(b) an antibody comprising a V_{H} comprising a sequence set forth in SEQ ID NO: 3 and a V_{L} comprising a sequence set forth in SEQ ID NO: 4;
(c) an antibody comprising a V_{H} comprising a sequence set forth in SEQ ID NO: 5 and a V_{L} comprising a sequence set forth in SEQ ID NO: 6;
(d) an antibody comprising a V_{H} comprising a sequence set forth in SEQ ID NO: 7 and a V_{L} comprising a sequence set forth in SEQ ID NO: 8; and
(e) an antibody comprising a V_{H} comprising a sequence set forth in SEQ ID NO: 9 and a V_{L} comprising a sequence set forth in SEQ ID NO: 10.

4. The protein of claim 1, wherein the antibody variable region cross-reacts with a peptide comprising a sequence set forth in SEQ ID NO: 51.

5. The protein of claim 1, wherein the protein comprises a fragment variable (Fv).

6. The protein of claim 5, wherein the protein is selected from the group consisting of:
(i) a single chain fragment variable (Fv) fragment (scFv);
(ii) a dimeric scFv (di-scFv);
(iii) a diabody;
(iv) a triabody;
(v) a tetrabody;
(vi) a fragment antigen binding (Fab);
(vii) a F(ab′)₂;
(viii) a Fv;
(ix) one of (i) to (viii) linked to a constant region of an antibody, a constant fragment (Fc) or a heavy chain constant domain (C_{H}) 2 and/or C_{H}3; and
(x) an antibody.

7. The protein of claim 1, wherein the protein is an antibody comprising:
(i) a V_{H} comprising a sequence set forth in SEQ ID NO: 45 and a V_{L} comprising a sequence set forth in SEQ ID NO: 46 or SEQ ID NO: 6; or
(ii) a V_{H} comprising three complementarity determining regions (CDRs) of a V_{H} comprising a sequence set forth in SEQ ID NO: 45 and a V_{L} comprising three CDRs of a V_{L} comprising a sequence set forth in SEQ ID NO: 46 or SEQ ID NO: 6; or
(iii) a V_{H} comprising a CDR1 comprising a sequence set forth in SEQ ID NO: 47, a CDR2 comprising a sequence set forth in SEQ ID NO: 48, and a CDR3 comprising a sequence set forth in SEQ ID NO: 49 and a V_{L} comprising a CDR1 comprising a sequence set forth in SEQ ID NO: 36 or SEQ ID NO: 32, a CDR2 comprising a sequence set forth in SEQ ID NO: 37 or SEQ ID NO: 33, and a CDR3 comprising a sequence set forth in SEQ ID NO: 38 or SEQ ID NO: 34.

8. The protein of claim 1, wherein the protein is an antibody comprising:
(i) a V_{H} comprising a sequence set forth in SEQ ID NO: 1 and a V_{L} comprising a sequence set forth in SEQ ID NO: 2;
(ii) a V_{H} comprising a sequence set forth in SEQ ID NO: 3 and a V_{L} comprising a sequence set forth in SEQ ID NO: 4;
(iii) a V_{H} comprising a sequence set forth in SEQ ID NO: 5 and a V_{L} comprising a sequence set forth in SEQ ID NO: 6;
(iv) a V_{H} comprising a sequence set forth in SEQ ID NO: 7 and a V_{L} comprising a sequence set forth in SEQ ID NO: 8; or
(v) a V_{H} comprising a sequence set forth in SEQ ID NO: 9 and a V_{L} comprising a sequence set forth in SEQ ID NO: 10.

9. The protein of claim 1, wherein the protein is an antibody comprising:
(i) a V_{H} comprising:
(a) a CDR1 comprising a sequence set forth in SEQ ID NO: 21;
(b) a CDR2 comprising a sequence set forth in SEQ ID NO: 22; and
(c) a CDR3 comprising a sequence set forth in SEQ ID NO: 23; and
a V_{L} comprising:
(a) a CDR1 comprising a sequence set forth in SEQ ID NO: 24;
(b) a CDR2 comprising a sequence set forth in SEQ ID NO: 25; and
(c) a CDR3 comprising a sequence set forth in SEQ ID NO: 26; or
(ii) a V_{H} comprising:
(a) a CDR1 comprising a sequence set forth in SEQ ID NO: 29;
(b) a CDR2 comprising a sequence set forth in SEQ ID NO: 30; and
(c) a CDR3 comprising a sequence set forth in SEQ ID NO: 35; and
a V_{L} comprising:
(a) a CDR1 comprising a sequence set forth in SEQ ID NO: 36;
(b) a CDR2 comprising a sequence set forth in SEQ ID NO: 37; and
(c) a CDR3 comprising a sequence set forth in SEQ ID NO: 38; or
(iii) a V_{H} comprising:
(a) a CDR1 comprising a sequence set forth in SEQ ID NO: 27;
(b) a CDR2 comprising a sequence set forth in SEQ ID NO: 28; and
(c) a CDR3 comprising a sequence set forth in SEQ ID NO: 35; and
a V_{L} comprising:
(a) a CDR1 comprising a sequence set forth in SEQ ID NO: 36;
(b) a CDR2 comprising a sequence set forth in SEQ ID NO: 437; and
(c) a CDR3 comprising a sequence set forth in SEQ ID NO: 38; or.
(iv) a V_{H} comprising:
(a) a CDR1 comprising a sequence set forth in SEQ ID NO: 329;
(b) a CDR2 comprising a sequence set forth in SEQ ID NO: 30; and
(c) a CDR3 comprising a sequence set forth in SEQ ID NO: 31; and
a V_{L} comprising:
(a) a CDR1 comprising a sequence set forth in SEQ ID NO: 32;
(b) a CDR2 comprising a sequence set forth in SEQ ID NO: 33; and
(c) a CDR3 comprising a sequence set forth in SEQ ID NO: 34; or
(v) a V_{H} comprising:
(a) a CDR1 comprising a sequence set forth in SEQ ID NO: 39;
(b) a CDR2 comprising a sequence set forth in SEQ ID NO: 40; and
(c) a CDR3 comprising a sequence set forth in SEQ ID NO: 41; and
a V_{L} comprising:
(a) a CDR1 comprising a sequence set forth in SEQ ID NO: 42;
(b) a CDR2 comprising a sequence set forth in SEQ ID NO: 43; and
(c) a CDR3 comprising a sequence set forth in SEQ ID NO: 44.

10.-12. (canceled)

13. A method of detecting an antigen having a conformation sufficient to induce an immune response against SARS-COV-2, the method comprising contacting a protein of claim 1 with the antigen and detecting binding of the protein to the antigen, wherein binding of the protein to the antigen indicates the antigen having a conformation sufficient to induce an immune response against SARS-COV-2.

14. (canceled)

15. The method of claim 13, wherein the antigen is a protein, a peptide, an attenuated virus or a virus-like particle.

16. (canceled)

17. (canceled)

18. A method of treating, preventing and/or delaying progression of a respiratory viral infection in a subject, the method comprising administering the protein of claim 1 to a subject in need thereof.

19. (canceled)

20. The method of claim 18, wherein the respiratory viral infection is selected from the group consisting of SARS-COV-2 infection, coronavirus disease 2019 (COVID-19) and acute respiratory disease syndrome (ARDS) and combinations thereof.

21. The method of claim 18, wherein a plurality of proteins, antibodies and/or pharmaceutical compositions are administered to the subject.

22. (canceled)

23. The protein of claim 1, wherein the protein is encoded by a polynucleotide comprising a nucleic acid sequence set forth in any one of SEQ ID NO: 11 to SEQ ID NO: 20.
